(19) United States
(12) Patent Application Publication
(10) Pub. No.: US 2026/0356894 A1
(43) Pub. Date: Nov. 12, 2026

(54) HETERODIMER OF POXVIRUS A16 AND G9 PROTEINS AS AN IMMUNOGEN

(71) Applicants: INSTITUT PASTEUR, PARIS 15 (FR); CENTRE NATIONAL DE LA RECHERCHE SCIENTIFIQUE, PARIS (FR)

(72) Inventors: Annalisa MEOLA, Paris (FR); Olivier SCHWARTZ, Paris (FR); Mathieu HUBERT, MAISONS ALFORT (FR); Pierre LAFAYE, Paris (FR); Pablo GUARDADO-CALVO, Paris (FR)

(21) Appl. No.: 19/483,795

(22) PCT Filed: May 17, 2024
(86) PCT No.: PCT/EP2024/063801

§ 371 Date: Nov. 12, 2025

(30) Foreign Application Priority Data
May 17, 2023 (EP) 23305795.9

(51) Int. Cl. C07K 14/005; A61K 39/00; A61P 31/20; C07K 14/47

(52) U.S. Cl. CPC C07K 14/005; A61P 31/20; C07K 14/4702; A61K 39/00; A61K 2039/575; C12N 2710/24122; C12N 2710/24134

(57) ABSTRACT

The invention relates to an isolated heterodimer of poxvirus A16 and G9 proteins as an immunogen in a subunit or nucleic acid vaccine against poxvirus. The invention provides an engineered heterodimer, nucleic acid encoding the heterodimer, neutralizing antibodies directed against said heterodimer and their use for the prevention, treatment and diagnostics of poxvirus infections and related diseases.

### FIELD OF THE INVENTION

The invention relates to an isolated heterodimer of poxvirus A16 and G9 proteins as an immunogen in a subunit or nucleic acid vaccine against poxvirus. The invention provides an engineered heterodimer, nucleic acid encoding the heterodimer, neutralizing antibodies directed against said heterodimer and their use for the prevention, treatment and diagnostics of poxvirus infections and related diseases.

### BACKGROUND OF THE INVENTION

Poxviruses are a family of enveloped viruses with a large dsDNA genome encoding for hundreds of proteins. Several members of this family cause significant human and animal diseases. The best known is variola virus (VARV), the etiological agent of smallpox that was eradicated 40 years ago after a worldwide vaccination campaign. Other relevant virus is monkeypox (MPOX), which produces recurrent epidemics in Africa. In 2022, a global mpox epidemy with more than 80,000 cases in 100 countries attracted worldwide attention and highlighted the need to develop new therapeutic tools to counter poxvirus diseases.

The first-generation vaccines used to eradicate smallpox used unattenuated vaccinia virus (VACV) produced in live animals. Over time, these vaccines were considered unsafe because of possible microbial contamination and the presence of allergenic animal proteins and were replaced by vaccines produced in tissue cultures or embryonated chicken eggs. These second-generation vaccines have a better profile but still produce severe adverse reaction in vaccinated people and, in absence of a global threat of poxvirus disease, its use is not recommended. Third-generation vaccines are based on the attenuated MVA (Modified Vaccinia virus Ankara). They have been approved by the FDA and the EMA and have been used to prevent mpox infections. Compared to second generation vaccines, they show a better safety profile but produce low levels of neutralizing antibodies against mpox. The three generations of vaccines mentioned above are based on vaccinia virus (virulent or attenuated). A general problem with all these VACV-based vaccines is that VACV produce hundreds of proteins, it is not known which and how many of them contribute to protection, but many have immunomodulatory properties whose impact on vaccinees is unknown.

One alternative is to produce subunit vaccines targeting specific antigens. These immunogens can be delivered as purified proteins or encoded in DNA, mRNA or viral vectors. There are two infectious poxvirus particles, the mature virion (MV) and the enveloped virion (EV). MVs are covered by a single membrane, released from the infected cell by lysis and play an important role in the dissemination between organisms. The membrane of MV display about 20 different proteins, 4 dedicated to bind cellular factors (D8, H3, A26, A27) and 11 required for viral fusion (A16, A21, A28, G3, G9, H2, J5, L5, O3, L1, F9). EVs are covered by an extra membrane, released from the infected cell by exocytosis and play a key role in dissemination within the organism. The external membrane of EV displays 4 proteins (A33, A34, B5, A56). So far, the neutralizing antibodies that have been identified target D8, H3, L1, A27, A33 and B5 but only those binding L1 neutralize the virus in the absence of complement, probably because they block the activity of the entry-fusion complex, which is essential for viral entry. To date, protective immunogens used in subunit vaccines have included MV proteins L1, A27, D8, and H3 and EV proteins B5 and A33 (Sakhatskyy et al., Virology, 2008, 5, 371; Golovkin et al., PNAS, 2007, 104, 6864-6869; Reeman et al., Viruses, 2017, 9, 378; Heraud et al., J. Immunol., 2006, 177, 2552-2564; WO 2005/013918; Zhang et al., Emerging Microbes & Infections, 2023, DOI: 10.1080/22221751.2023.2192815).

The fusion complex of poxvirus is different from any other described to date and by far more complex. Poxviruses code for 11 proteins dedicated to mediated membrane fusion, grouped in a single complex called the entry fusion complex (EFC). Nine proteins form the core of the EFC (A16, A21, A28, G3, G9, H2, J5, L5, O3) to which two others (L1, F9) are associated peripherally. All of them contain a transmembrane region and most have conserved disulphide bonds catalyzed by virus-encoded thiol oxidoreductases within the cytoplasm of infected cells. They are highly conserved across the family (>95% identity between VACV and MPOX). Assuming each component is present only once in the complex (the actual stoichiometry of the complex is not known) the estimated molecular mass of the complex would be about 250 kDa. Genetic repression of EFC subunits results in the formation of morphologically normal virions that are incompetent for fusion and in which the EFC is delocalized and scattered in several stable subcomplexes, among which the heterocomplexes formed by A28:H2, A16:G9 and G3:L5. So far, only the crystal structures of the isolated L1 and F9 have been reported. In addition to 11 proteins to catalyze fusion, poxviruses have 3 additional proteins (A26, A56, K2) to regulate them, the so-called viral suppressors.

New protective immunogens of poxvirus would be useful to fight poxvirus infections and related diseases.

### SUMMARY OF THE INVENTION

Despite the complexity associated with the expression of transmembrane, non-glycosylated proteins having conserved disulphide bonds catalysed by virus-encoded enzymes within the cytoplasm of infected cells (Table 1), the inventors have engineered a heterodimer of poxvirus A16 and G9 recombinant proteins that folds as in the fusion complex on the viral surface (FIG. 4). Furthermore, they have shown that surprisingly, the recombinant A16/G9 heterodimer is able to induce neutralizing antibodies against poxvirus (FIG. 5). Due to the high degree of conservation of the A16 and G9 proteins in poxviruses (FIGS. 2 and 3), the A16/G9 heterodimer may be used as a vaccine against various poxvirus diseases. Co-expression of A16 and G9 nucleic acid constructs in cell form a well-folded heterodimer able to produce neutralizing antibodies. Therefore, the heterodimer of poxvirus A16 and G9 proteins may be used as an immunogen in a subunit or nucleic acid vaccine against poxvirus.

The invention relates to an isolated heterodimer of poxvirus A16 and G9 proteins or nucleic acid encoding said heterodimer for use as a vaccine.

In some embodiments of the vaccine according to the invention, the heterodimer or nucleic acid induces neutralizing antibodies against poxvirus.

In some embodiments of the vaccine according to the invention, the A16 and G9 proteins are from orthopoxvirus; preferably chosen from: Vaccinia virus, Monkeypox, Ectromelia virus, Variola virus, Cowpoxvirus, Camelpox virus, and Tatera poxvirus.

In some embodiments of the vaccine according to the invention, the heterodimer comprises truncated A16 and G9 proteins comprising or consisting of the ectodomain or a fragment thereof comprising the beta-folded head domain and small alpha helices rich domain. Preferably, the truncated A16 and G9 proteins comprise or consist of an amino acid sequence selected from the group consisting of:
a) a sequence having at least 85% identity with the sequence from position 1 to any one of positions 333 to 343 of SEQ ID NO: 1; preferably having at least 85% identity with the sequence from position 1 to positions 338 of SEQ ID NO: 1;
b) a sequence having at least 85% identity with the sequence from position 1 to any one of positions 287 to 297 of SEQ ID NO: 1; preferably having at least 85% identity with the sequence from position 1 to position 295 of SEQ ID NO: 1;
c) a sequence having at least 85% identity with the sequence from position 1 to any one of positions 316 to 326 of SEQ ID NO: 8; preferably having at least 85% identity with the sequence from position 1 to position 321 of SEQ ID NO: 1; and
d) a sequence having at least 85% identity with the sequence from position 1 to any one of positions 263 to 273 of SEQ ID NO: 8; preferably having at least 85% identity with the sequence from position 1 to position 271 of SEQ ID NO: 1.
   More preferably, the heterodimer comprises a truncated A16 protein as defined in a) and a truncated G9 protein as defined in c) or a truncated A16 protein as defined in b) and a truncated G9 protein as defined in d); still more preferably the heterodimer comprises a truncated A16 protein as defined in a) and a truncated G9 protein as defined in c).

In some embodiments of the vaccine according to the invention, the A16 and G9 proteins are produced from a precursor comprising a N-terminal signal peptide.

In some embodiments of the vaccine according to the invention, the A16 and G9 proteins comprise the mutation of the N-terminal myristoylation motif; preferably comprising the G2A substitution.

In some embodiments of the vaccine according to the invention, the A16 and G9 proteins comprise the mutation of one or more or all of the N-glycosylation sites at position N315 of A16 protein and at positions N82, N93, N154, N157 and N273 of G9 protein; preferably wherein the A16 protein comprises the T317A substitution and the G9 protein comprises one or more or all of the substitutions N82A, N93Q, S156A, N157D and S275N, said positions being determined by alignment with A16 of SEQ ID NO: 1 and G9 of SEQ ID NO: 8.

In some particular embodiments of the vaccine according to the invention, the truncated A16 and G9 proteins comprise or consist of an amino acid sequence chosen from any one of SEQ ID NO: 15 to 18; preferably the heterodimer comprises the pair of sequences SEQ ID NO: 15 and SEQ ID NO: 16 or SEQ ID NO: 17 and SEQ ID NO: 18; more preferably SEQ ID NO: 15 and SEQ ID NO: 16.

In some particular embodiments of the vaccine according to the invention, the A16 and G9 proteins are recombinant proteins.

In some particular embodiments of the vaccine according to the invention, the nucleic acid is mRNA, preferably modified mRNA, which is enclosed in a particle or vesicle, preferably lipid nanoparticle (LNP).

In some particular embodiments of the vaccine according to the invention, the nucleic acid is inserted into a viral vector, preferably selected from the group consisting of: adenovirus, adeno-associated virus, vesicular stomatitis virus, lentivirus, poxvirus such as vaccinia virus including modified vaccinia virus Ankara, insect-specific viral vectors, and combinations thereof.

In some embodiments, the vaccine according to the invention is for use in the prevention or treatment of Monkeypox virus infection and related disease.

Another aspect of the invention relates to an engineered heterodimer or nucleic acid encoding said heterodimer according to the present disclosure.

Another aspect of the invention relates to the in vitro use of the heterodimer according to the present disclosure for poxvirus diagnostics.

The invention also relates to a neutralizing antibody against poxvirus, which is directed against the heterodimer, preferably engineered heterodimer, according to the present disclosure.

### DETAILED DESCRIPTION OF THE INVENTION

The invention provides an isolated heterodimer of poxvirus A16 and G9 proteins which induces neutralizing antibodies against poxvirus and is useful as an immunogen in a subunit or nucleic acid vaccine against poxvirus infections and related diseases. The invention also provides an engineered heterodimer. that folds as in the fusion complex on the viral surface. The invention further provides poxvirus neutralizing antibodies directed against the heterodimer, in particular engineered heterodimer, and their use for the treatment of poxvirus infections and related diseases. The invention also encompasses the use of the heterodimer, in particular engineered heterodimer, and neutralizing antibodies for poxvirus diagnostics.

### Heterodimer and Use as an Immunogen in a Vaccine Against Poxvirus

The invention relates to an isolated heterodimer of poxvirus A16 and G9 proteins which induces neutralizing antibodies against poxvirus and is used as an immunogen in a subunit or nucleic acid vaccine against poxvirus infections and related diseases.

The isolated heterodimer of poxvirus A16 and G9 proteins according to the invention is named poxvirus A16/G9 heterodimer, poxvirus heterodimer, A16/G9 heterodimer or heterodimer. The heterodimer produced from recombinant A16 and G9 proteins is named recombinant A16/G9 heterodimer or recombinant heterodimer.

The heterodimer according to the invention folds as native A16 and G9 proteins on the viral surface and induces neutralizing antibodies against poxvirus as disclosed in the examples of the present application.

An immunogen as used herein, refers to a substance able to elicit an immune response when introduced into a subject. An immunogen according to the invention is a protective immunogen, able to induce neutralizing antibodies against poxvirus. The induction of neutralizing antibodies by the poxvirus A16/G9 heterodimer may be determined by standard virus neutralization assays that are well-known in the art such as those disclosed in the present examples.

The isolated heterodimer of the invention is a complex of two proteins, A16 and G9. It is produced by co-expression of poxvirus A16 and G9 proteins using recombinant or synthetic nucleic acid constructs encoding said proteins and therefore does not comprise any other protein(s) of the entry fusion complex (EFC).

A heterodimer of recombinant A16 and G9 proteins may be produced in an appropriate expression system to generate a sub-unit vaccine. Alternatively, a nucleic acid encoding the A16/G9 heterodimer or vector comprising said nucleic acid is produced to generate a nucleic acid vaccine.

Nomenclatures of A16 and G9 refer to vaccinia virus a prototype and representative member of the poxvirus family (named Poxviridae) which includes four genera that infect humans: Orthopoxvirus, Parapoxvirus, Yatapoxvirus and Molluscipoxvirus. Orthopoxvirus include smallpox virus (VARV or variola), vaccinia virus (VACV), cowpox virus (CPXV), monkeypox virus (MPXV); Parapoxvirus include orf virus, pseudocowpox, bovine papular stomatitis virus; Yatapoxvirus include tanapoxvirus, yaba monkey tumor virus; Molluscipoxvirus include molluscum contagiosum virus (MCV). Smallpox (variola) has been declared eradicated in 1979. The most common diseases in human are monkeypox infections which are rising. Ectromelia virus (ECTV) is a mouse-specific orthopoxvirus that causes mousepox. Camelpox virus (CMLV) is a camel-specific orthopoxvirus that causes a wide-spread infectious viral disease in camels and dromaderies. Tatera poxvirus (TATV), isolated from a wild gerbil, is the closest related virus to variola. squirrelpox virus (SQPV) is a member of the genus sciuripoxvirus that causes fatal disease squirrelpox in United Kingdom red squirrels.

Vaccinia virus A16 and G9 have the amino acid sequences of SEQ ID NO: 1 and 8, respectively. Other representative examples are shown in FIGS. 2 and 3 and include without limitation A16 homologs of: Monkeypox virus (SEQ ID NO: 7); Ectromelia virus (SEQ ID NO: 6); Variola virus (SEQ ID NO: 5); Cowpoxvirus (SEQ ID NO: 4); Camelpox virus (SEQ ID NO: 2) and Tatera poxvirus (SEQ ID NO: 3) and G9 homologs of Ectromelia virus (SEQ ID NO: 14); Cowpoxvirus (SEQ ID NO: 13); Camelpox virus (SEQ ID NO: 12); Monkeypox virus (SEQ ID NO: 11); Tatera poxvirus (SEQ ID NO: 9) and Variola virus (SEQ ID NO: 10).

Based on structure prediction (FIG. 1) and confirmation by crystallographic studies (FIG. 4) both A16 and G9 proteins can be divided into four distinct regions: a beta-folded head domain (indicated by an H in the FIG. 1), a domain rich in small alpha helices, termed the body (B, in the Figure), a domain that forms a long 2-strand beta sheet, consisting of 1 strand of A16 and 1 strand of G9, which is called the tail (T), and a transmembrane region (TM). A cytoplasmic (CT) domain is also found in A16.

In the protein A16, the H domain is from positions M1 to D134; the B domain is from positions Y135 to K292; the T domain is from positions Y293 to K338; the TM and cytoplasmic (CT) domains are from positions L339 to R377 of SEQ ID NO: 1 and include the TM domain from positions L339 do S363 and the cytoplasmic domain from position R364 to R377. The extracellular domain or ectodomain (E) is from positions M1 to K338 of SEQ ID NO: 1. A16 protein comprises a N-terminal myristoylation motif MG and a N-glycosylation site NCT in N315.

In the protein G9, the H domain is from positions M1 to P59; the B domain is from positions D60 to N268; the T domain is from positions 1269 to K321 and the TM domain is from positions L322 to 1340 of SEQ ID NO: 8. The ectodomain (E) is from positions: M1 to K321 of SEQ ID NO: 8. G9 protein comprises a N-terminal myristoylation motif MG. G9 protein also comprises five N-glycosylation sites: NGT in N82; NRT in N93; NRS in N154; NRT in N157 and NTS in N283.

The above-indicated positions of the different structural domains of A16 and G9 proteins may vary by few amino acids without altering the overall structure of the A16/G9 heterodimer. Therefore, the invention encompasses domains which differ from the indicated positions by the addition or deletion of up to 5 consecutive amino acids.

In the following description, the residues are designated by the standard one letter amino acid code and the indicated positions are determined by alignment with SEQ ID NO: 1 for A16 or SEQ ID NO: 8 for G9. Substitutions are designated herein by the one letter amino acid code followed by the substituting residue in one letter amino acid code; G2A is a substitution of the Glycine (G) residue at position 2 of SEQ ID NO: 1 or 8 with a Alanine (A) residue. One skilled in the art can easily determine the positions in another A16 or G9 protein, by alignment with the reference sequence using appropriate software available in the art such as BLAST, CLUSTALW and others. [0037]“a”, “an”, and “the” include plural referents, unless the context clearly indicates otherwise. As such, the term “a” (or “an”), “one or more” or “at least one” can be used interchangeably herein; unless specified otherwise, “or” means “and/or”.

The term “subject” or “individual” refers to both human and non-human animals. Preferably, a subject according to the invention is a human.

### Heterodimer of A16 and G9 Proteins, in Particular Engineered Heterodimer

The poxvirus A16/G9 heterodimer according to the invention may be derived from A16 and G9 proteins of any poxvirus, including variants of poxvirus A16 and G9 proteins. In some embodiments, the A16/G9 heterodimer according to the invention is derived from A16 and G9 proteins of an orthopoxvirus or variants thereof. In particular embodiments, the A16/G9 heterodimer is derived from A16 and G9 proteins of an orthopoxvirus chosen from: Vaccinia virus (VACV); Monkeypox virus (MPXV); Ectromelia virus (ECTV); Variola virus (VARV); Cowpoxvirus (CPXV); Camelpox virus (CMLV); Taterapox virus (TATV) and variants thereof; particularly Vaccinia virus (VACV) or variants thereof. The A16/G9 heterodimer may be derived from A16 of any one of SEQ ID NO: 1 to 7 or a variant thereof; and from G9 of any one of SEQ ID NO: 2 and 8 to 12 or a variant thereof. The heterodimer preferably comprises A16 and G9 proteins derived from the same poxvirus, in particular vaccinia virus or variants thereof.

As used herein, the term “variant” refers to a polypeptide comprising an amino acid sequence having at least 70% sequence identity with the native sequence; preferably having at least 75%, 80% or 85% sequence identity with the native sequence. The term “variant” refers to a functional variant having the activity of the native sequence. The activity of a variant may be assessed using methods well-known by the skilled person such as those disclosed herein.

As used herein, the term “functional variant”, refers to a A16 or G9 variant that forms a functional heterodimer that folds as native A16 and G9 proteins on the viral surface and induces neutralizing antibodies against poxvirus as disclosed in the examples of the present application.

As used herein, the term “functional heterodimer”, refers to a heterodimer that folds as native A16 and G9 proteins on the viral surface and induces neutralizing antibodies against poxvirus as disclosed in the examples of the present application.

The percent amino acid sequence or nucleotide sequence identity is defined as the percent of amino acid residues or nucleotides in a Compared Sequence that are identical to the Reference Sequence after aligning the sequences and introducing gaps if necessary, to achieve the maximum sequence identity and not considering any conservative substitutions for amino acid sequences as part of the sequence identity. Alignment for purposes of determining percent amino acid sequence identity can be achieved in various ways known to a person of skill in the art, for instance using publicly available computer software such as the GCG (Genetics Computer Group, Program Manual for the GCG Package, Version 7, Madison, Wisconsin) pileup program, or any of sequence comparison algorithms such as BLAST (Altschul et al., J. Mol. Biol., 1990, 215, 403-), FASTA or CLUSTALW. When using such software, the default parameters, are preferably used. The BLASTP program uses as default a word length (W) of 3 and an expectation (E) of 10.

In some embodiments, the term “variant” refers to a polypeptide having an amino acid sequence that differs from a native sequence by the substitution, insertion and/or deletion of less than 100, 90, 80, 70, 60, 50, 40, 30, 25, 20, 15, 10 or 5 amino acids. In a preferred embodiment, the variant differs from the native sequence by one or more conservative substitutions, preferably by less than 50, 40, 30, 25, 20, 15, 10 or 5 conservative substitutions. Conservative substitutions are substitutions of one amino acid with another having similar chemical or physical properties (size, charge or polarity), which substitution generally does not adversely affect the biochemical, biophysical and/or biological properties of the protein. Examples of conservative substitutions may be within the following groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W); or within the groups of basic amino acids (R, K, H), acidic amino acids (D, E), polar amino acids (Q, N), hydrophobic amino acids (M, L, I, V), aromatic amino acids (F, W, Y), and small amino acids (G, A, S, T).

In some embodiments, the heterodimer is an engineered heterodimer comprising modified A16 and G9 proteins (i.e. A16 and G9 variants) having mutations (insertion, deletion, substitution) which allow the expression of proteins that form a well-folded complex and are able to produce neutralizing antibodies against poxvirus.

In particular embodiments, the engineered heterodimer comprises A16 and G9 variants having mutations which target proteins to the secretory pathway, such as a N-terminal signal peptide or signal sequence. The lumen of the endoplasmic reticulum is the good place to form disulphide bonds that are involved in proper folding of the A16 and G9 proteins. Signal peptides (SP) are short peptide sequences which are present at the N-terminus of secretory proteins and are used to target proteins for secretion. Signal peptides are removed from the mature protein during this process by a specific peptidase. Therefore, the signal peptide is present in the protein precursor but not in the secreted protein. Multiple signal peptides are known in the art and publicly available (see in particular, Signal Peptide Website and SPdb sequence databases). Signal peptides useful in the present invention include in particular those from the following proteins: Drosophila melanogaster Hsc70-3(BiP) corresponding to the sequence SEQ ID NO: 19, IL-2 corresponding to the sequence SEQ ID NO: 22; preferably the signal peptide comprises or consists of SEQ ID NO: 19.

To produce a secreted heterodimer, truncated A16 and G9 proteins lacking the transmembrane domain are engineered. As used herein, a secreted heterodimer refers to a heterodimer which is transported outside the cell after synthesis. A secreted heterodimer can be recovered in the cell supernatant. The truncated proteins A16 and G9 proteins comprise a Cter deletion of the TM domain (G9) or TM and CT domains (A16). In particular embodiments, the engineered heterodimer comprises truncated A16 and G9 proteins comprising or consisting of the ectodomain or a fragment thereof comprising the beta-folded head domain (H domain) and small alpha helices rich domain (body or B domain).

In more particular embodiments, the truncated A16 protein comprises the Ectodomain (E) and lacks the TM and CT domains. Said truncated A16 protein may comprise or consist of the sequence from position 1 to any one any one of positions 333 to 343 of SEQ ID NO: 1 or a variant thereof preferably from positions 1 to 338 of SEQ ID NO: 1 or a variant thereof. In some embodiments, the truncated A16 protein comprises a C-terminally truncated A16 amino acid sequence having at least 70%, 75%, 80% or more identity with the sequence from position 1 to any one of positions 333 to 343 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to any one of positions 333 to 343 of SEQ ID NO: 1. In some particular embodiments, the truncated A16 protein comprises a C-terminally truncated A16 amino acid sequence having at least 70%, 75%, 80% or more identity with the sequence from position 1 to position 338 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to position 338 of SEQ ID NO: 1. In some preferred embodiments, the truncated A16 protein comprises or consists of the sequence SEQ ID NO: 15.

In more particular embodiments, the truncated A16 protein comprises a fragment of the Ectodomain comprising the H and B domains and lacks the TM and CT domains. Said truncated A16 protein may comprise or consist of the sequence from position 1 to any one any one of positions 287 to 297 of SEQ ID NO: 1 or a variant thereof preferably from positions 1 to 295 of SEQ ID NO: 1 or a variant thereof. In some embodiments, the truncated A16 protein comprises a C-terminally truncated A16 amino acid sequence having at least 70%, 75%, 80% or more identity with the sequence from position 1 to any one of positions 287 to 297 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to any one of positions 287 to 297 of SEQ ID NO: 1. In some particular embodiments, the truncated A16 protein comprises a C-terminally truncated A16 amino acid sequence having at least 70%, 75%, 80% or more identity with the sequence from position 1 to position 295 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to position 295 of SEQ ID NO: 1. In some preferred embodiments, the truncated A16 protein comprises or consists of the sequence SEQ ID NO: 17.

In more particular embodiments, the truncated G9 protein comprises the Ectodomain (E) and lacks the TM domain. Said truncated G9 protein may comprise or consist of the sequence from position 1 to any one any one of positions 316 to 326 of SEQ ID NO: 8 or a variant thereof preferably from positions 1 to 321 of SEQ ID NO: 8 or a variant thereof. In some embodiments, the truncated G9 protein comprises a C-terminally truncated G9 amino acid sequence having at least 70%, 75%, 80% or more identity with the sequence from position 1 to any one of positions 316 to 326 of SEQ ID NO: 8; preferably at least 85%, 86%, 87%, 88%, 89%, 90%, 91%, 92%, 93%, 94%, 95%, 96%, 97% or 98% identity with the sequence from position 1 to any one of positions 316 to 326 of SEQ ID NO: 8. In some particular embodiments, the truncated G9 protein comprises a C-terminally truncated G9 amino acid sequence having at least 70%, 75%, 80% or more identity with the sequence from position 1 to position 321 of SEQ ID NO: 8; preferably at least 85%, 86%, 87%, 88%, 89%, 90%, 91%, 92%, 93%, 94%, 95%, 96%, 97% or 98% identity with the sequence from position 1 to position 321 of SEQ ID NO: 8. In some preferred embodiments, the truncated G9 protein comprises or consists of the sequence SEQ ID NO: 16.

In more particular embodiments, the truncated G9 protein comprises a fragment of the Ectodomain comprising the H and B domains and lacks the TM domain. Said truncated G9 protein may comprise or consist of the sequence from position 1 to any one any one of positions 263 to 273 of SEQ ID NO: 8 or a variant thereof preferably from positions 1 to 271 of SEQ ID NO: 8 or a variant thereof. In some embodiments, the truncated G9 protein comprises a C-terminally truncated G9 amino acid sequence having at least 70%, 75%, 80% or more identity with the sequence from position 1 to any one of positions 263 to 273 of SEQ ID NO: 8; preferably at least 85%, 86%, 87%, 88%, 89%, 90%, 91%, 92%, 93%, 94%, 95%, 96%, 97% or 98% identity with the sequence from position 1 to any one of positions 263 to 273 of SEQ ID NO: 8. In some particular embodiments, the truncated G9 protein comprises a C-terminally truncated G9 amino acid sequence having at least 70%, 75%, 80% or more identity with the sequence from position 1 to position 271 of SEQ ID NO: 8; preferably at least 85%, 86%, 87%, 88%, 89%, 90%, 91%, 92%, 93%, 94%, 95%, 96%, 97% or 98% identity with the sequence from position 1 to position 271 of SEQ ID NO: 8. In some preferred embodiments, the truncated G9 protein comprises or consists of the sequence SEQ ID NO: 18.

In particular embodiments, the N-terminal myristoylation motif is mutated (i.e. inactivated), for example by the G2A substitution. In more particular embodiments, the A16 and/or G9 proteins comprise the G2A substitution.

The native A16 and G9 viral proteins are not naturally glycosylated despite the presence of glycosylation motifs. Glycosylation that would occur by directing the A16 and G9 proteins to the secretory pathway may have a detrimental impact on the folding and immunological properties of the proteins. Therefore, in particular embodiments, the A16 and G9 proteins comprise mutated (i.e. inactivated) glycosylation motif(s), in particular mutated N-glycosylation motif(s) to mimic native viral proteins that are not naturally glycosylated. In more particular embodiments, the A16 protein comprises the mutation of the N-glycosylation site at N315; preferably comprising the mutation T317A. In more particular embodiments, the G9 protein comprises the mutation of one or more or all of the N-glycosylation sites at positions N82, N93, N154, N157 and N273; preferably comprising one or more or all of the mutations N82A, N93Q, S156A, N157D and S275N.

The A16 and G9 proteins may further comprise an additional heterologous sequence (other than the N-terminal signal peptide). The heterologous sequence is a sequence different from the sequence naturally present in the native A16 and G9 sequence. It is usually of up to 50 amino acids. In some embodiments, the heterologous sequence is added at the C-terminus of the A16 and/or G9 sequence. In some embodiments, the added heterologous sequence is a tag, in particular a purification tag suitable for affinity purification such as polyhistidine tag or streptavidine tag including double streptavidine tag. Polyhistidine tag usually comprises at least 5 histidines which bind to metal matrices comprising nickel or cobalt. Streptavidin tag binds specifically to ligands such as streptactin. The tag may be removable by chemical agents or by enzymatic means such as proteases (TEV protease, Thrombin, Factor Xa or Enteropeptidase). In some particular embodiments, the tag comprises or consists of the sequence: SEQ ID NO: 20 or SEQ ID NO: 21. In some particular embodiments, A16 comprises a C-terminal Histidine tag, preferably comprising the sequence SEQ ID NO: 20. In some particular embodiments, G9 comprises a C-terminal streptavidine tag, preferably comprising the sequence SEQ ID NO: 21. The tags of SEQ ID NO: 20 and 21 are cleavable tags that can be removed by enzymatic cleavage with thrombin.

In some preferred embodiments, the heterodimer is made of recombinant A16 and G9 proteins as disclosed herein; preferably recombinant truncated A16 and G9 proteins as disclosed herein (i.e., comprising the Ectodomain (E) or a fragment thereof comprising the H and B domains and lacking the TM domain (G9) or TM and CT domains (A16)); more preferably comprising the Ectodomain (E) and lacking the TM domain (G9) or TM and CT domains (A16); more preferably recombinant truncated A16 and G9 proteins further comprising the mutation of the N-terminal myristoylation and/or the mutation of one or more of all of the N-glycosylation sites as disclosed herein.

A first type of heterodimer is made of recombinant truncated A16 and G9 proteins comprising a fragment of the Ectodomain (E) comprising the H and B domains and lacking the TM domain (G9) or TM and CT domains (A16) as disclosed herein. The recombinant truncated A16 protein of said heterodimer may comprise or consist of the sequence from position 1 to any one any one of positions 287 to 297 of SEQ ID NO: 1 or a variant thereof preferably from positions 1 to 295 of SEQ ID NO: 1 or a variant thereof. In some embodiments, the recombinant truncated A16 protein of said heterodimer comprises a C-terminally truncated A16 amino acid sequence having at least 70%, 75%, 80% or more identity with the sequence from position 1 to any one of positions 287 to 297 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to any one of positions 287 to 297 of SEQ ID NO: 1. In some particular embodiments, the recombinant truncated A16 protein of said heterodimer comprises a C-terminally truncated A16 amino acid sequence having at least 70%, 75%, 80% or more identity with the sequence from position 1 to position 295 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to position 295 of SEQ ID NO: 1. The recombinant truncated G9 protein of said heterodimer may comprise or consist of the sequence from position 1 to any one any one of positions 263 to 273 of SEQ ID NO: 8 or a variant thereof preferably from positions 1 to 271 of SEQ ID NO: 8 or a variant thereof. In some embodiments, the recombinant truncated G9 protein of said heterodimer comprises a C-terminally truncated G9 amino acid sequence having at least 70%, 75%, 80% or more identity with the sequence from position 1 to any one of positions 263 to 273 of SEQ ID NO: 8; preferably at least 85%, 86%, 87%, 88%, 89%, 90%, 91%, 92%, 93%, 94%, 95%, 96%, 97% or 98% identity with the sequence from position 1 to any one of positions 263 to 273 of SEQ ID NO: 8. In some particular embodiments, the recombinant truncated G9 protein of said heterodimer comprises a C-terminally truncated G9 amino acid sequence having at least 70%, 75%, 80% or more identity with the sequence from position 1 to position 271 of SEQ ID NO: 8; preferably at least 85%, 86%, 87%, 88%, 89%, 90%, 91%, 92%, 93%, 94%, 95%, 96%, 97% or 98% identity with the sequence from position 1 to position 271 of SEQ ID NO: 8. In some preferred embodiments, the recombinant truncated A16 and G9 proteins of said heterodimer further comprise the mutation of the N-terminal myristoylation, in particular G2A mutation. In some more preferred embodiments, the recombinant truncated G9 protein of said heterodimer further comprises the mutation of the N-glycosylation sites at positions N82, N93, N154, N157 and optionally N273 if present; preferably comprising the mutations N82A, N93Q, S156A, and N157D. In some even more preferred embodiments, the recombinant truncated A16 protein of said heterodimer comprises or consists of the sequence SEQ ID NO: 17 and the recombinant truncated G9 protein of said heterodimer comprises or consists of the sequence SEQ ID NO: 18.

A preferred type of heterodimer is made of recombinant truncated A16 and G9 proteins comprising the Ectodomain (E) and lacking the TM domain (G9) or TM and CT domains (A16) as disclosed herein. The recombinant truncated A16 protein of said heterodimer may comprise or consist of the sequence from position 1 to any one any one of positions 333 to 343 of SEQ ID NO: 1 or a variant thereof preferably from positions 1 to 338 of SEQ ID NO: 1 or a variant thereof. In some embodiments, the recombinant truncated A16 protein of said heterodimer comprises a C-terminally truncated A16 amino acid sequence having at least 70%, 75%, 80% or more identity with the sequence from position 1 to any one of positions 333 to 343 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to any one of positions 333 to 343 of SEQ ID NO: 1. In some particular embodiments, the recombinant truncated A16 protein of said heterodimer comprises a C-terminally truncated A16 amino acid sequence having at least 70%, 75%, 80% or more identity with the sequence from position 1 to position 338 of SEQ ID NO: 1; preferably at least 85%, 86%, 87%, 88%, 89%, 90%, 91%, 92%, 93%, 94%, 95%, 96%, 97%, 98% or 99% identity with the sequence from position 1 to position 338 of SEQ ID NO: 1. The recombinant truncated G9 protein of said heterodimer may comprise or consist of the sequence from position 1 to any one any one of positions 316 to 326 of SEQ ID NO: 8 or a variant thereof preferably from positions 1 to 321 of SEQ ID NO: 8 or a variant thereof. In some embodiments, the recombinant truncated G9 protein of said heterodimer comprises a C-terminally truncated G9 amino acid sequence having at least 70%, 75%, 80% or more identity with the sequence from position 1 to any one of positions 316 to 326 of SEQ ID NO: 8; preferably at least 85%, 86%, 87%, 88%, 89%, 90%, 91%, 92%, 93%, 94%, 95%, 96%, 97% or 98% identity with the sequence from position 1 to any one of positions 316 to 326 of SEQ ID NO: 8. In some particular embodiments, the recombinant truncated G9 protein of said heterodimer comprises a C-terminally truncated G9 amino acid sequence having at least 70%, 75%, 80% or more identity with the sequence from position 1 to position 321 of SEQ ID NO: 8; preferably at least 85%, 86%, 87%, 88%, 89%, 90%, 91%, 92%, 93%, 94%, 95%, 96%, 97% or 98% identity with the sequence from position 1 to position 321 of SEQ ID NO: 8. In some preferred embodiments, the recombinant truncated A16 and G9 proteins of said heterodimer further comprise the mutation of the N-terminal myristoylation, in particular G2A mutation. In some more preferred embodiments, the recombinant truncated A16 protein of said heterodimer further comprises the mutation of the N-glycosylation site at N315; preferably comprising the mutation T317A and the recombinant truncated G9 protein of said heterodimer further comprises the mutation of the N-glycosylation sites at positions N82, N93, N154, N157 and N273; preferably comprising the mutations N82A, N93Q, S156A, N157D and S275N. In some even more preferred embodiments, the truncated A16 protein of said heterodimer comprises or consists of the sequence SEQ ID NO: 15 and the truncated G9 protein of said heterodimer comprises or consists of the sequence SEQ ID NO: 16.

### Nucleic Acid and Vector Encoding Heterodimer

The invention relates also to an isolated nucleic acid comprising a nucleotide sequence encoding the heterodimer as disclosed herein; preferably encoding an engineered heterodimer as disclosed herein; preferably comprising nucleotide sequences encoding the truncated A16 and G9 proteins as disclosed herein; more preferably comprising nucleotide sequences encoding the truncated A16 and G9 proteins further comprising the mutation of the N-terminal myristoylation and/or the mutation of one or more of all of the N-glycosylation sites as disclosed herein.

The nucleic acid may be recombinant, synthetic or semi-synthetic nucleic acid which is expressible in a host cell suitable for protein expression or production. The host cell may a cell for recombinant protein production (subunit vaccine) or a subject cell for protein production in vivo (nucleic acid vaccine). The nucleic acid may be DNA, RNA, or mixed molecule, either single- and/or double-stranded which may further be modified and/or included in any suitable expression vector. As used herein, the terms “vector” and “expression vector” mean the vehicle by which a DNA or RNA sequence (e.g. a foreign gene) can be introduced into a host cell, so as to transform the host and promote expression (e.g. transcription and translation (DNA) or translation (RNA)) of the introduced sequence. The recombinant vector can be a vector for eukaryotic or prokaryotic expression. An expression vector as defined herein is chosen to enable the production of a protein, either in vitro or in vivo.

The nucleic acid may comprise a coding sequence which is optimized for the cell in which the heterodimer is expressed. The coding sequences for the A16 and G9 proteins may be on the same nucleic acid molecule or on separate nucleic acid molecules.

In some embodiments, said nucleic acid comprises at least a coding sequence for the A16 and/or G9 proteins selected from the group consisting of: SEQ ID NO: 23 to 26; preferably the pair of coding sequences SEQ ID NO: 23 and 24 or SEQ ID NO: 25 and 26. SEQ ID NO: 23 encodes the truncated A16 protein of SEQ ID NO: 15; SEQ ID NO: 24 encodes the truncated G9 protein of SEQ ID NO: 16; SEQ ID NO: 25 encodes the truncated A16 protein of SEQ ID NO: 17; SEQ ID NO: 26 encodes the truncated G9 protein of SEQ ID NO: 18.

In some embodiments, said nucleic acid is an expression cassette, preferably a eukaryote expression cassette, wherein the A16 and/or G9 coding sequence(s) is operably linked to appropriate regulatory sequence(s) for their expression in a protein producing cell or a subject cell. Such sequences which are well-known in the art include in particular a promoter, and further regulatory sequences capable of further controlling the expression of a transgene, such as without limitation, enhancer or activator, terminator, Kozak sequence and intron (in eukaryote), ribosome-binding site (RBS) (in prokaryote). In some particular embodiments, the coding sequence is operably linked to a promoter. The promoter may be a ubiquitous, constitutive or inducible promoter that is functional in the cell. Such promoters are well-known in the art and their sequences are available in public sequence data bases. In some embodiments, the expression cassette is a mammalian expression cassette for expression of the A16 and/or G9 proteins in a subject cell. In some other embodiments, the expression cassette is an insect cell expression cassette for expression of the A16 and/or G9 proteins in insect cells.

In some embodiments, the A16 and G9 proteins are co-expressed in the cell using a nucleic acid molecule encoding both A16 and G9 proteins. The nucleic acid molecule may comprise two separate expression cassettes for the A16 and G9 proteins. Alternatively, the A16 and G9 proteins may be expressed from a unique expression cassette using an Internal Ribosome Entry Site (IRES) inserted between the two coding sequences or a viral 2A.

In some embodiments, the nucleic acid is RNA, preferably mRNA, wherein the coding sequence of the A16 and/or G9 protein is operably linked to appropriate regulatory sequence(s) for their expression in an individual's target cells or tissue(s). mRNA therapy is well-known in the art. mRNA is delivered into the host cell cytoplasm where expression generates the therapeutic protein of interest. mRNA construct comprises a cap structure, 5′ and 3′untranslated regions (UTRs), and open reading frame (ORF), and a 3′poly(A) tail. mRNA construct may be non-replicating mRNA (MRM) or self-amplifying mRNA (SAM). SAM comprises the inclusion of genetic replication machinery derived from positive-strand mRNA viruses, most commonly alphaviruses such as Sindbis and Semliki-Forest viruses. In SAM constructs, the ORF encoding viral structural protein is replaced by the transcript encoding the therapeutic protein of interest, and the viral RNA-dependent RNA polymerase is retained to direct cytoplasmic amplification of the replicon construct. Trans-replicating RNA are disclosed for example in WO 2017/162461. RNA replicon from alphavirus suitable for gene expression are disclosed in WO 2017/162460. mRNA manufacturing process uses plasmid DNA (pDNA) containing a DNA-dependent RNA polymerase promoter, such as T7, and the corresponding sequence for the mRNA construct. The pDNA is linearized to serve as a template for the DNA-dependent RNA polymerase to transcribe the mRNA, and subsequently degraded by a DNase process step. The addition of the 5′cap and the 3′poly(A) tail can be achieved during the in vitro transcription step or enzymatically after transcription. Enzymatic addition of the cap can be accomplished by using guanylyl transferase and 2′-O-methyltransferase to yield a Cap0(^{N7Me}GpppN) or Cap1 (^{N7Me}GpppN^{2′-oMe}) structure, respectively, while the poly-A tail can be achieved through enzymatic addition via poly-A polymerase. mRNA is then purified using standard methods suitable for mRNA purification such as high-pressure liquid chromatography (HPLC) and others. Methods for producing mRNA are disclosed for example in WO 2017/182524.

To improve translation efficiency in treated subject cells, the mRNA according to the invention comprises a sequence which is codon-optimized for expression in human. Further improvements of the mRNA construct according to the invention to improve its stability and translation efficiency in vivo include optimization the length and regulatory element sequences of 5′-UTR and 3′UTR; base and/or sugar modifications in the cap structure to increase ribosomal interaction and/or mRNA stability; and modified nucleosides. Modified nucleosides may be in the 5′-UTR, 3′-UTR or ORF. Examples of modified nucleosides include pseudouridine and N-1-methylpseudouridine that remove intracellular signalling triggers for protein kinase R activation. Examples of modified nucleosides that reduce RNA degradation into cells are disclosed in WO 2013/039857. Modified cap structures are disclosed in WO 2011/015347 and WO 2019/175356. Optimized 3′-UTR sequences are disclosed in WO 2017/059902. Modified polyA sequences which improve RNA stability and translation efficiency are disclosed in US 2020/0392518. Modified mRNA with improved stability and translation efficiency are also disclosed in WO 2007/036366.

The invention also relates to a vector comprising the nucleic acid construct according to the present disclosure. The invention may use any vector suitable for the delivery and expression of nucleic acid (DNA or RNA sequence, e.g. a foreign gene) into cells. In some embodiments, the vector is suitable for expression of recombinant proteins in appropriate protein producing cell. In some embodiments, the vector is suitable for expression of proteins in a subject cell, in particular suitable for vaccination. Such vectors that are well-known in the art include viral and non-viral vectors. In addition, these approaches can advantageously be combined to introduce and maintain the nucleic acid of the invention into cells.

Non-viral vector includes the various (non-viral) agents which are commonly used to either introduce or maintain nucleic acid into a cell. Agents which are used to introduce nucleic acid into a cell by various means include in particular polymer-based, particle-based, lipid-based, peptide-based delivery vehicles or combinations thereof, such as with no limitations cationic polymer, dendrimer, micelle, liposome, lipopolyplex, exosome, microparticle and nanoparticle including lipid nanoparticle (LNP) and viral-like particles; and cell penetrating peptides (CPP). Agents which are used to maintain nucleic acid into cells so as to transform the host and promote expression (e.g. transcription and translation for DNA and translation for RNA) of the introduced sequence are recombinant vectors or expression vectors. Such vectors include in particular naked nucleic acid vectors such as plasmids, transposons and mini-circles. The vector may be a replicating vector such as a replicating plasmid. The replicating vector such as replicating plasmid may be a low-copy or high-copy number vector or plasmid. These vectors may have minimal eukaryotic sequences to minimize the possibility of chromosomal integration.

Viral vectors are by nature capable of penetrating into cells and deliver nucleic acid(s) of interest into cell, according to a process named as viral transduction. In viral vectors, viral genes essential for replication and virulence are replaced with an expression cassette for the transgene of interest. Thus, the viral vector genome comprises the transgene expression cassette flanked by the viral sequences required for viral vector production. Viral vectors suitable as vaccine vector (viral vector vaccine) include in particular adenovirus (Ad), adeno-associated virus (AAV), vesicular stomatitis virus (VSV), lentivirus, poxvirus such as vaccinia virus including modified vaccinia virus Ankara, insect-specific viral vectors, and combinations thereof (For a review, see Travieso et al., npj Vaccines, 2022, 7, 75). Heterologous viral vector vaccine (HVVV) consists of a combination of two or more vectors encoding either the same or different antigens that can be administered together or in prime boost regimens. HVVV immunizations can be advantageous to induce higher magnitude and more durable immune responses compared to homologous vector vaccination. Viral expression vectors suitable for recombinant protein production include in particular baculovirus.

In some embodiments, the vector is a vaccine vector for the delivery and expression of the heterodimer according to the present disclosure in a subject cell. In some particular embodiments, the vaccine vector comprises mRNA, preferably modified mRNA, encoding the heterodimer according to the present disclosure enclosed in a particle or vesicle, in particular lipid-based micro- or nano-vesicle or particle such as liposome or lipid nanoparticle (LNP). In some particular embodiments, the vaccine vector comprises nucleic acid encoding the heterodimer according to the present disclosure inserted in a viral vector. In more particular embodiments, the nucleic acid is DNA. In more particular embodiments, the viral vector is selected from the group consisting of: adenovirus (Ad), adeno-associated virus (AAV), vesicular stomatitis virus (VSV), lentivirus, poxvirus such as vaccinia virus including modified vaccinia virus Ankara, insect-specific viral vectors, and combinations thereof.

In some embodiments, the vector is an expression vector for the recombinant production of the heterodimer according to the present disclosure in a protein producing cell. Host cell may be prokaryote, in particular a bacterial cell such as E. coli cell. Preferably, host cell is eukaryote, including mammalian and insect cells. In some particular embodiments, the vector is a mammalian expression plasmid or a baculovirus; preferably a baculovirus.

The nucleic acid according to the invention is prepared by the conventional methods known in the art. For example, it is produced by amplification of a nucleic sequence by PCR or RT-PCR, by screening genomic DNA libraries by hybridization with a homologous probe, or else by total or partial chemical synthesis. The recombinant vectors are constructed and introduced into host cells by the conventional recombinant DNA techniques, which are known in the art.

A further object of the present disclosure relates to a host cell which has been transfected, infected or transformed by a nucleic acid and/or a vector according to the invention. As used herein, the term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The transformation may be transient or stable over time. Stable transformation may be by integration of the nucleic acid into the host cell genome. A host cell that receives and expresses introduced DNA or RNA has been “transformed”. Preferably, host cell is eukaryote, including mammalian and insect cells.

Another aspect of the invention relates to a method of production of the engineered heterodimer according to the present disclosure, comprising: (i) culturing the host cell of the present disclosure for expression of said engineered heterodimer by the host cell; (ii) recovering the engineered heterodimer from the culture medium or host cells; and (iii) purifying said engineered heterodimer.

The invention provides the use of the heterodimer as a vaccine (nucleic acid or sub-unit vaccine) as disclosed herein. The invention also provides an engineered heterodimer as disclosed herein, wherein the heterodimer comprises modified A16 and G9 proteins (i.e A16 and G9 variants) having mutations (insertion, deletion, substitution) which allow the expression of proteins that form a well-folded complex and are able to produce neutralizing antibodies against poxvirus as disclosed herein and illustrated in the examples of the present application. The invention also provides a nucleic acid encoding the engineered heterodimer as disclosed herein.

### Immunogenic or Vaccine Composition and Therapeutic Use

Another aspect of the invention relates to an immunogenic or vaccine pharmaceutical composition comprising, as active substance (i.e., immunogen), an isolated heterodimer or a nucleic acid or derived vector encoding said heterodimer according to the present disclosure; and at least one pharmaceutically acceptable vehicle. The pharmaceutical composition, heterodimer, nucleic acid or vector according to the present disclosure are used for preventing or treating a poxvirus infection and/or associated disease in a subject in need thereof.

The pharmaceutical vehicles are those appropriate to the planned route of administration, which are well known in the art.

The pharmaceutical composition may further comprise an adjuvant. Non-limitative examples of adjuvants suitable for use in the composition of the invention include various delivery systems, immune potentiators, combined adjuvants and mucosal adjuvants such as: microparticles including uni- or multi-lamellar liposomes, ISCOMS, virosomes, viral pseudo-particules, micelles, saccharid (poly(lactide-co-glycolide)), gold microspheres, and nanoparticules; CpG oligodeoxynucleotide; polyI:C (polyinosinc-polycytidylic acid); oil emulsion such as Freund's adjuvants, MF59, ASO3; mineral substances such as aluminium salts; bacterial extracts; Flagellin; saponin; monophosphoryl-lipid A; squalene; L-pampo, MALP-2, Pam2CSK4 and Pam3CSK4; Imiquimod and resiquimod, AS01, ASO4, ASO2, Cholera toxin (CT), Heat-labile enterotoxin (LTK3 and LTR72); chitosan (Review in Facciola et al., Vaccines, 2022, 10, 819).

The pharmaceutical composition comprises a therapeutically effective amount of the heterodimer, nucleic acid or derived vector encoding the heterodimer sufficient to induce a protective immune response against poxvirus infection in the individual to whom it is administered. The protective immune response includes the production of neutralizing antibodies against poxvirus. The pharmaceutically effective dose depends upon the composition used, the route of administration, the physical characteristics of the specific human under consideration, concurrent medication, and other factors, that those skilled in the medical arts will recognize.

The invention provides also an isolated heterodimer or nucleic acid or derived vector encoding the heterodimer according to the present disclosure for use as a medicament.

The invention provides also an isolated heterodimer or nucleic acid or vector encoding the heterodimer according to the present disclosure or pharmaceutical composition according to the present disclosure for use in the prevention or treatment of poxvirus infection and related disease.

The invention provides also a method for preventing or treating poxvirus infection and associated disease, comprising: administering a therapeutically effective amount of the pharmaceutical composition according to the invention to a subject.

The invention provides also the use of an heterodimer according to the present disclosure or pharmaceutical composition according to the present disclosure for the prevention or treatment of poxvirus infection and related disease.

The invention provides also the use of an heterodimer according to the present disclosure in the manufacture of a medicament for the prevention or treatment of poxvirus infection and related disease.

The invention provides also a pharmaceutical composition for the prevention or treatment of poxvirus infection and related disease, comprising an heterodimer according to the present disclosure as an active component.

The invention provides also a pharmaceutical composition comprising an heterodimer according to the present disclosure for preventing or treating poxvirus infection and related disease.

The infection is preferably caused by an orthopoxvirus, in particular monkeypox virus.

The pharmaceutical composition of the present invention is generally administered according to known procedures, at dosages and for periods of time effective to induce a beneficial effect in the subject. The administration may be oral, by injection such as subcutaneous or intramuscular, or mucosal administration such as intranasal.

### Neutralizing Antibody Directed Against the Heterodimer and Use Thereof

Another aspect of the invention relates to a poxvirus neutralizing antibody which is directed against the isolated heterodimer, preferably an engineered heterodimer, according to the present disclosure.

The poxvirus neutralizing activity of the antibody according to the invention may be determined by standard virus neutralization assays that are well-known in the art such as those disclosed in the present examples. In particular, the antibody according to the invention as a neutralizing activity against monkeypox virus and/or vaccinia virus.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies. The term “antibody” herein is used in the broadest sense and includes polyclonal and monoclonal antibodies, including intact antibodies and functional (antigen-binding) antibody fragments, including fragment antigen binding (Fab) fragments; Fab′, Fab′-SH, F(ab′)2 fragments; Fv fragments; recombinant IgG (rIgG) fragments; variable heavy chain (VH) regions capable of specifically binding the antigen; single chain antibody fragments, including single chain variable fragments (scFv), and single domain antibodies (e.g., sdAb, sdFv, nanobody or VHH) fragments. The term encompasses antibodies comprising a heavy chain variable region and a light chain variable region as well as antibodies comprising only a heavy chain (Heavy chain only antibody or HcAb). The term also encompasses intact or full-length antibodies, including antibodies of any class or sub-class, including IgG and sub-classes thereof, IgG1, IgG2, IgG3, IgG4, IgM, IgE, IgA, and IgD. The term encompasses recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof.

By the term “specifically binds to” or “is specific for” as used herein with respect to an antibody, is meant an antibody or antibody fragment, which recognizes and binds to a specific antigen, but does not recognize or bind in significant amount (detectably bind) to other molecules present in the sample or to other molecules to which the antibody may come into contact in an organism, while detectably binding to its specific antigen. The specific binding of an antibody to its antigen may be determined by standard assays such as immunoassay. An antibody specifically binds to its antigen when it has a dissociation constant (KD) of 1 μM or less for its antigen in a standard KD determination assay. KD values are expressed as molar concentration (M); KD for antibodies are usually determined by Surface plasmon resonance using Biacore assay.

“recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

In some embodiments, the antibody is a heavy chain only antibody or an antigen-binding fragment thereof (nanobody or VHH) or a Fc fusion thereof (Ig-VHH). In some other embodiment, the neutralizing antibody is a monoclonal antibody, preferably a human or humanised monoclonal antibody.

The neutralizing antibody may be produced by standard antibody production techniques that are well-known in the art (Review in Sanlav et al., J. Basic Clin. Health Sci., 2020, 4, 197-204). For example, it may be produced by immunization of an animal with the heterodimer according to the present disclosure, as disclosed in the examples of the present application. Antibodies of the present disclosure can be produced in a host cell transfectoma using, for example, a combination of recombinant DNA techniques and gene transfection methods as is well known in the art (Morrison, 1985).

Another aspect of the invention relates to a pharmaceutical composition comprising, as active substance a neutralizing antibody according to the present disclosure; and at least one pharmaceutically acceptable vehicle.

The pharmaceutical vehicles are those appropriate to the planned route of administration, which are well known in the art.

The pharmaceutical composition comprises a therapeutically effective amount of the sufficient to limit the viral infection in the subject. The pharmaceutically effective dose depends upon the composition used, the route of administration, the physical characteristics of the specific human under consideration, concurrent medication, and other factors, that those skilled in the medical arts will recognize.

The invention provides also a neutralizing antibody according to the present disclosure for use as a medicament.

The invention provides also a neutralizing antibody according to the present disclosure or pharmaceutical composition according to the present disclosure for use in the prevention or treatment of poxvirus infection and related disease.

The invention provides also a method for preventing or treating poxvirus infection and associated disease, comprising: administering a therapeutically effective amount of the pharmaceutical composition according to the invention to a subject.

The invention provides also the use of a neutralizing antibody according to the present disclosure or pharmaceutical composition according to the present disclosure for the prevention or treatment of poxvirus infection and related disease.

The invention provides also the use of a neutralizing antibody according to the present disclosure in the manufacture of a medicament for the prevention or treatment of poxvirus infection and related disease.

The invention provides also a pharmaceutical composition for the prevention or treatment of poxvirus infection and related disease, comprising a neutralizing antibody according to the present disclosure as an active component.

The invention provides also a pharmaceutical composition comprising a neutralizing antibody according to the present disclosure for preventing or treating poxvirus infection and related disease.

The infection is preferably caused by an orthopoxvirus, in particular monkeypox virus.

The pharmaceutical composition of the present invention is generally administered according to known procedures, at dosages and for periods of time effective to induce a beneficial effect in the subject. The administration may be oral, by injection such as subcutaneous or intramuscular, or mucosal administration such as intranasal.

### Use of Heterodimer or Neutralizing Antibody Thereto for Poxvirus Diagnostics

The isolated heterodimer (antigen) and neutralizing antibody thereto according to the present disclosure are useful as reagent for the detection or diagnosis of poxvirus.

The invention relates to the in vitro use of the heterodimer antigen or neutralizing antibody thereto according to the present disclosure as reagent for the detection or diagnosis of poxvirus.

The invention also relates to a method of detection or diagnosis of poxvirus, comprising determining the presence of A16 and/or G9 protein of said virus or antibodies thereto in a sample using the heterodimer antigen or neutralizing antibody thereto according to the present disclosure.

The detection or diagnosis is generally performed by immunoassay. Immunoassays are well-known techniques for protein or antibody detection which rely on the detection of antigen-antibody complexes using an appropriate label. The method of the invention may use any immunoassay such as with no limitations, immunoblotting, immunoprecipitation, ELISA, immunocytochemistry or immunohistochemistry, and immunofluorescence like flow cytometry assay, and FACS. The method of the invention may use any appropriate label used in immunoassays such as enzymes, biotin, fluorescent dyes/proteins or others.

In some embodiments, the method of detection or diagnosis of poxvirus infection, comprises the step of:
incubating the heterodimer antigen or antibody thereto according to the present disclosure with the biological sample to form a mixture; and,
detecting antigen-antibody complexes in the mixture.

The sample for poxvirus antibody detection is preferably body fluid from the individual, in particular serum.

The antigen or antibody is preferably labeled and the antigen-antibody complexes are detected by measuring the signal from the label by any appropriate means available for that purpose as disclosed above.

The detection of the antigen-antibody complexes in a sample from the individual indicates that the individual is suffering from poxvirus past or present infection.

In some embodiments in connection with this aspect of the invention, the above method comprises a further step of administering an appropriate treatment to the individual depending on whether or not the individual is diagnosed with poxvirus virus infection and in particular with a disease caused by poxvirus.

Another aspect of the invention is a kit for the diagnosis or detection of poxvirus, comprising at least one heterodimer antigen or antibody thereto, as defined above, preferably further including a detectable label.

The practice of the present invention will employ, unless otherwise indicated, conventional techniques which are within the skill of the art. Such techniques are explained fully in the literature.

The invention will now be exemplified with the following examples, which are not limitative, with reference to the attached drawings in which:

### FIGURE LEGENDS

FIG. 1. Alphafold model of the complex A16/G9. A16 is depicted in dark gray and G9 in light gray. The four domains are indicated as superscripts: Head (H), Body (B), Tail (T) and the transmembrane helices (TM). The position of the sequons are indicated with green (G9) and cyan (A16) spheres.

FIG. 2. Alignment of A16 homologs in the genus orthopoxvirus MPXV: Monkeypox virus (SEQ ID NO: 7). ECTV: Ectromelia virus (SEQ ID NO: 6). VARV: Variola virus (SEQ ID NO: 5). CPXV: Cowpoxvirus (SEQ ID NO: 4). VACV: Vaccinia virus (SEQ ID NO: 1). CMLV: Camelpox virus (SEQ ID NO: 2). TATV: Tatera poxvirus (SEQ ID NO: 3).

FIG. 3. Alignment of G9 homologs in the genus orthopoxvirus ECTV: Ectromelia virus (SEQ ID NO: 14). CPXV: Cowpoxvirus (SEQ ID NO: 13). CMLV: Camelpox virus (SEQ ID NO: 12). MPXV: Monkeypox virus (SEQ ID NO: 11). VACV: Vaccinia virus (SEQ ID NO: 8). TATV: Tatera poxvirus (SEQ ID NO: 9). VARV: Variola virus (SEQ ID NO: 10).

FIG. 4. Production of A16:G9 A) Crystal structure of the complex A16:G9 from Squirrelpox virus where it can be seen that the two N-terminal ends cluster together at one side of the complex. The crystal structure confirms that the prediction of the alphafold complex is generally correct. B) SDS-PAGE of the purified A16:G9 complex which confirms the presence of both proteins of the complex. C) Sensorgrams showing the interaction of A56:K2 with A16:G9. The left panel is the interaction of the VACV fusion suppressor (A56/K2) with the VACV A16^{E}/G9^{E} heterodimer, the right panel is a control showing that the VACV suppressor cannot interact with the squirrelpox A16^{E}/G9^{E} heterodimer. This experiment shows that the heterodimer produced is folded in the same way as on the viral surface.

FIG. 5. Neutralization of MVA-GFP and MPXV by the immune serum from A16^{E}/G9^{E}-immunized lama. A) MVA-GFP or MPXV were mixed to serial dilutions of a serum obtained from an alpaca immunized with an irrelevant protein (Control) or with the A16^{E}/G9^{E} complex (A16^{E}/G9^{E}). The mixture was then incubated for 30 min at room temperature (RT) and added onto target cells. After 24 to 48 h, the cells were fixed. MVA-GFP infection was monitored by measuring the GFP signal while MPXV infection was monitored by immunofluorescence using a rabbit polyclonal anti-VACV antibody. B) MVA-GFP neutralization assay. Left: examples of micrographs. Right: quantification of the neutralization and determination of the median effective dose (ED50) as the dilution of serum necessary to reduce MVA-GFP infection by 50%, using the “No serum” condition as the basis for maximal infection. C) MPXV neutralization assay. Left: examples of micrographs. Right: heatmap showing the presence (+, red) or the absence (−, blue) of a neutralizing activity by considering as neutralization every well in which the MPXV signal is below that of the “No serum” condition. The neutralizing titer was determined by selecting the highest dilution in which neutralization was observed and was expressed as the corresponding dilution factor.

FIG. 6. Nanobodies targeting A16/G9 neutralize different orthopoxviruses. Neutralization assays with and without complement of the four IgG-VHHs using VACV (top panel) and MPXV (bottom panels). Two neutralizing antibodies that do not require complement to neutralize the virus: 7D11 (α-L1, Su et al., Virology, 2007, 368, 331-341) and VACV-302 (α-A27, Gilchuk et al., Cell, 2016, 167, 684-694) were included as control.

### EXAMPLES

### Material and Methods

### Protein Expression and Purification

In order to obtain milligrams amount of soluble A16/G9, a synthetic gene codon-optimized for expression in Drosophila cells was inserted into a modified, bicistronic pMT/BiP plasmid (Invitrogen). These constructs included the full length A16/G9 ectodomain (A16^{E}/G9^{E}), i.e. lacking the transmembrane and cytoplasmatic domains, or short versions (A16^{HB}/G9^{HB}), in which the tail regions of both proteins have been removed. To help in the purification two strep-tag sequences separated by a flexible linker and a His-tag sequence, both preceded by a thrombin cleave site, were included in the C-terminus of G9 and A16, respectively. In addition, some mutations were also introduced to remove the N-glycosylation and myristoylation motifs. These plasmids were used to obtain stable transfectants of Drosophila S2 cells together with the pCoPuro plasmid (ratio 1:20) for puromycin selection. The stable cell lines were selected and maintained in serum-free Insect-Xpress medium containing 7 μg/ml puromycin. Cultures of 1-3 liters were grown in spinner flasks in Insect-Xpress medium supplemented with 1% penicillin/streptomycin antibiotics to about 1×10⁷ cells/mL, and the protein expression was induced with 4 μM CdCl₂. After 5 days, the S2 media supernatant was concentrated to 40 ml and supplemented with 10 μg/mL avidin and 0.1M Tris-HCl pH 8.0, centrifuged 30 minutes at 20,000 g and purified by strep-tactin and Ni-NTA affinity chromatography followed by gel filtration. The yields were about 5-10 mg/L.

### Immunization of Animals and Neutralization Experiments

One young adult male alpaca (Lama pacos) was immunized at days 0, 17 and 24 with 150 μg of recombinant A16^{E}/G9^{E}, in which the purification tags have been removed by targeted proteolysis with thrombin. The immunogen was mixed with Freund complete adjuvant for the first immunization and with Freund incomplete adjuvant for the following immunizations. The immune response was monitored by titration of serum samples by ELISA on coated A16^{E}/G9^{E}. The blood of the immunized animal was collected and the neutralization activity of the serum measured using a microneutralization assay with MVA (Modified vaccinia virus) virus carrying a GFP reported gene (MVA-GFP) and a monkeypox strain (MPXV/2022/FR/CMIP) isolated from a pustular lesion of French man in June 2022.

### Alpaca Immunization, Library Construction and Phage Display

One young adult male alpaca (Lama pacos) received immunizations on days 0, 17, and 24 with 150 μg of thrombin-digested rA16^{E}/G9^{E}, i.e., recombinant protein without tags. The immunogen was mixed with Freund complete adjuvant for the initial immunization and with Freund incomplete adjuvant for subsequent immunizations. The immune response was monitored by titrating serum samples using ELISA with the coated antigen and polyclonal rabbit anti-alpaca IgG. The blood of the immunized animal (approximately 200 ml) was collected, and peripheral blood lymphocytes were isolated by centrifugation on a Ficoll discontinuous gradient (Leucosep Tubes, Greiner) and stored at −80 °C until further use. Total RNA and cDNA were obtained as previously described (Gransagne, JBC, doi: 10.1016/j.jbc.2021.101290) and a nested PCR was performed with IgG-specific primers (Moeglin, Cancers 2021, doi.org/10.3390/cancers13133317). In the first step, five sets ofPCR primers were used to amplify the VH-CH1-CH2 and VHH-CH2 fragments. The bands corresponding to the VHH-CH2 regions were purified on an agarose gel. Next, VHH regions were specifically reamplified with three sets of VHH-specific PCR primers complementary to the 5′ and 3′ ends of the amplified product and incorporating Sfil and Notl restriction sites at the ends of the VHH genes. The PCR products were then digested and ligated into a pHEN6 phagemid vector.

Phage display technology allows the selection of antigen specific phage-VHHs. A large number of phage-VHHs (1013) were used to perform three rounds of panning. Pannings was performed as follows: phages-VHHs were incubated with strep-tagged rA16/G9 for 1 hour at room temperature and the complex was trapped using streptactin beads (Mag step XT beads, IBA)). 100 μM of strep-tagged rA16/G9 was used for the first round of panning, 10 μM and 1 μM of strep-tagged protein respectively for the second and third round of pannings. To remove nonspecific binders, 6 washes with PBS Tween 0.10% and 4 washes with PBS were performed, specific phage-VHHs were then eluted in 100 mM triethylamine (TEA) for 5 min on a wheel, and the excess TEA was neutralized immediately with 1 M Tris-HCl, pH 7.6. E. coli TG1 at exponential growth phase were then infected with eluted phage-VHHs and incubated at 37 °C for 30 min without stirring and 30 min under stirring. The bacteria were spread on a 2YT+ampicillin Bio-assay dish (24 cm×24 cm) and incubated overnight at 30 °C.

Phage-VHHs were produced from individual colonies, and binding of the phages to the protein on plate was revealed with an anti-M13 monoclonal antibody conjugated to peroxidase (Abcam). The VHH nucleotide sequences were determined using M13-40 primer (Eurofins).

### Production of VHHs and IgG-VHHs

To express monomeric VHHs in bacteria, codon-optimized sequences of the selected VHHs were cloned into the bacterial expression vector pET28c(+) (Novagen) with a C-terminal His tag. E. coli BL21 (DE3) cells (New England Biolabs) were transformed and induce protein expression overnight at 16 °C with 0.5 mM isopropyl β-d-1-thiogalactopyranoside (IPTG). Cells harvested from 3 l of culture were resuspended in 40 mL cold resuspension buffer (PBS pH 7.4, 5 mM Imidazole) supplemented by one tablet of complete protease inhibitor (Roche), frozen at −20 °C and lysed using a sonicator. After removing the insoluble material by centrifugating at 20,000 g for 20 minutes, the recombinant VHHs were purified using TALON affinity columns followed by SEC with a Superdex 75 column (Cytiva). To express the dimeric VHH in mammalian cells, codon-optimized sequences were inserted into a mammalian expression vector pCAGGS (ENA code: LT727518.1) in frame with an Fc at the C-terminal end. 100 mL of Expi293F™ cells (Thermo Fischer) were transiently transfected at a density of 3×10⁶ cells/ml using FectroPRO DNA transfection reagent (Polyplus). After 5 days incubation at 37 °C, the supernatant was harvested and centrifuged (30 minutes, 4000 rpm). Antibodies were purified using protein G affinity chromatography, eluted using 0.1 M glycine pH 2.5 and neutralized with 1 M Tris-HCl pH 8.0. The eluate was concentrated and used to perform a size exclusion chromatography in phosphate buffer saline (PBS) pH=7.4

### MVA-GFP Neutralization Assay

The assay is disclosed in Hubert et al., Cell Host and Microbe, 2023, DOI: 10.1016/j;chom.2023.05.001. Vero E6 cells were plated at 2×10⁴ cells per well in a Clear 96-well plate (Greiner Bio-One). MVA-GFP were mixed (ratio 1:1) with serial dilutions (from 1/30 to 1/7680) of serum (from an animal immunized with A16^{E}/G9^{E} and a control animal) in the presence of 10% guinea pig serum as a source of complement (GPC; Rockland). After incubation for 2 h at 37 °C, the mixture was added onto Vero E6 cell monolayers. Twenty hours later, cells were fixed for 30 min at room temperature (RT) with 4% paraformaldehyde (PFA, Electron Microscopy Sciences), washed and stained with Hoechst (1:1,000 dilution; Invitrogen). Images were acquired with an Opera Phenix high-content confocal microscope (Perkin Elmer). The GFP area and the number of nuclei were quantified using the Harmony software (Perkin Elmer). The percentage of neutralization was calculated using the GFP area as the value with the following formula: 100×(1−(value with serum−value in “noninfected”)/(value in “no serum”-value in “noninfected”)). Neutralizing activity was expressed as the IC50 (effective dose inhibiting 50% of infection). IC50 were calculated using a reconstructed curve with the percentage of neutralization at the different serum concentrations.

### Monkeypox Neutralization Assay

U2OS cells were plated at 2×10⁴ cells per well in a Clear 96-well plate (Greiner Bio-One). MPXV were mixed (ratio 1:1) in a BSL-3 facility with serial dilutions of serum from an animal immunized with A16^{E}/G9^{E} and a control animal or monoclonal antibodies in the presence 10% GPC. After incubation for 2 h at 37 °C, the mixture was added onto U2OS cells monolayers. Forty-eight hours later, cells were fixed for 30 min at RT with 4% paraformaldehyde, washed and immunostained for MPXV antigens with rabbit polyclonal anti-VACV antibodies (PA1-7258, Invitrogen), and an Alexa Fluor 488-coupled goat anti-rabbit antibody (Invitrogen). Nuclei were stained with Hoechst. Images were acquired with an Opera Phenix high-content confocal microscope (PerkinElmer). The MPXV+ area and the number of nuclei were quantified using the Harmony software (PerkinElmer). The neutralization titer was determined as the highest plasma, serum or monoclonal antibody dilution in which the MPXV+ area was inferior to that of the control serum, as indicated.

### VACV Neutralization Assay

BSC-40 cells were plated at 2×10⁴ cells per well in a black 96-well plate with clear bottom (Greiner Bio-One). 1000 PFU of VACV (Western Reserve strain) encoding for a GFP reporting gene (VACV-GFP) were mixed (ratio 1:1) with serial dilutions of IgG-VHHs in the presence or absence of 1% guinea pig serum as a source of complement (GPC; Rockland). After incubation for 2 h at 37 °C, the mixture was added onto BSC-40 cell monolayers. Twenty-four hours later, cells were fixed for 30 min at room temperature (RT) with 4% paraformaldehyde (PFA, Electron Microscopy Sciences). Images were acquired with a FluoroSpot (ImmunoSpot, CTL). The number of green foci was quantified using the FluoroSpot proprietary software and the percentage of neutralization was calculated as follow: 100−100×(N−Nn)/(Np−Nn) where N is the number of foci in the treatment, Nn is the number of spots detected in non-infected cells and Np is the number of foci detected in non-treated infected cells. Neutralizing activity of each IgG-VHH was expressed as the IC50 (effective dose inhibiting 50% of infection). IC50 values were calculated using a reconstructed curve with the percentage of neutralization at the antibody concentrations using Prism software (Version 10.2.1).

### Results

Because it is highly conserved and exert a key role in viral entry, the entry-fusion complex is the ideal target for developing subunit vaccines. However, its huge complexity prevents it from being easily expressed and purified. After considering each of the components of the complex, several lines of evidence suggest that the sub-complex A16:G9, which accounts for more than 1/3 of the total mass of the EFC (Table 1), is the ideal target.

TABLE 1
Entry-fusion complex proteins

| | | | | |
|---|---|---|---|---|
| | MW | Nb. of | Nb. of | N-ter |
| Protein | (kDa) | disulphide | sequons | myr |

Core proteins

| | | | | |
|---|---|---|---|---|
| A28 | 16 | 2 | 3 | No |
| H2 | 21 | 2 | 0 | No |
| L5 | 15 | 1 | 1 | No |
| A21 | 13 | 2 | 1 | No |
| G3 | 13 | 0 | 0 | No |
| O3 | 4 | 0 | 1 | No |
| A16 | 43 | 10 | 1 | Yes |
| G9 | 38 | 7 | 5 | Yes |
| J5 | 15 | 4 | 3 | No |

Associated proteins

| | | | | |
|---|---|---|---|---|
| L1 | 27 | 3 | 3 | Yes |
| F9 | 24 | 3 | 1 | No |
| Total | 230 kDa | | | |

A16 and G9 are paralogs well conserved through the family. Indeed, it is possible to identify homologues in other giant DNA viruses, like African Swine Fever Virus. This suggests that all these proteins evolved from a common ancestor and play and essential role in all these DNA viruses. Antibodies that bind to these proteins could easily interfere with its function and prevent viral fusion. In line with this hypothesis, viral suppressors block fusion by physically binding the sub-complex A16:G9.

However, expressing recombinant A16 and G9 is complicated. All of the EFC components are transmembrane, non-glycosylated proteins and most of them have conserved disulphide bonds catalysed by virus-encoded enzymes within the cytoplasm of infected cells (Table 1). In the absence of these enzymes, producing these proteins in the cytoplasm of a cell is not feasible. An alternative approach is to include a signal peptide at the N-terminal end of the proteins and direct them to the secretory pathway. The advantage of this approach is that the lumen of the endoplasmic reticulum is the good place to form disulphide bonds. The disadvantage is that none of the proteins of the EFC are naturally glycosylated but many contain glycosylation motifs (sequons). Then, an unmodified version of the protein would be glycosylated, which can have a major impact in the folding and in the immunological properties of the protein. The challenge is to modify both proteins in such a way that they can pass through the secretory pathway without being glycosylated. They should form a well folded complex and be able to produce neutralizing antibodies.

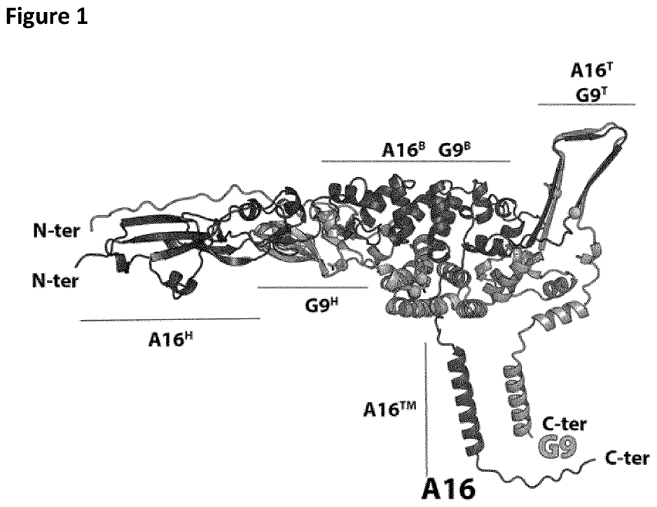

To do this, the inventors have identified all the glycosylation sites and mapped them onto a 3D model of the A16/G9 complex that were produced using alphafold (FIG. 1). Structurally, both A16 and G9 can be divided into four distinct regions: a beta-folded head domain (indicated by an H in the FIG. 1), a domain rich in small alpha helices, which is termed the body (B, in the Figure), a domain that forms a long 2-strand beta sheet, consisting of 1 strand of A16 and 1 strand of G9, which is called the tail (T), and a transmembrane region (TM). Of the 6 sequons, 4 are in the domain B and 2 in the domain T, 3 of them are close to the dimerization interface and could potentially prevent heterodimer formation.

### Construct Design

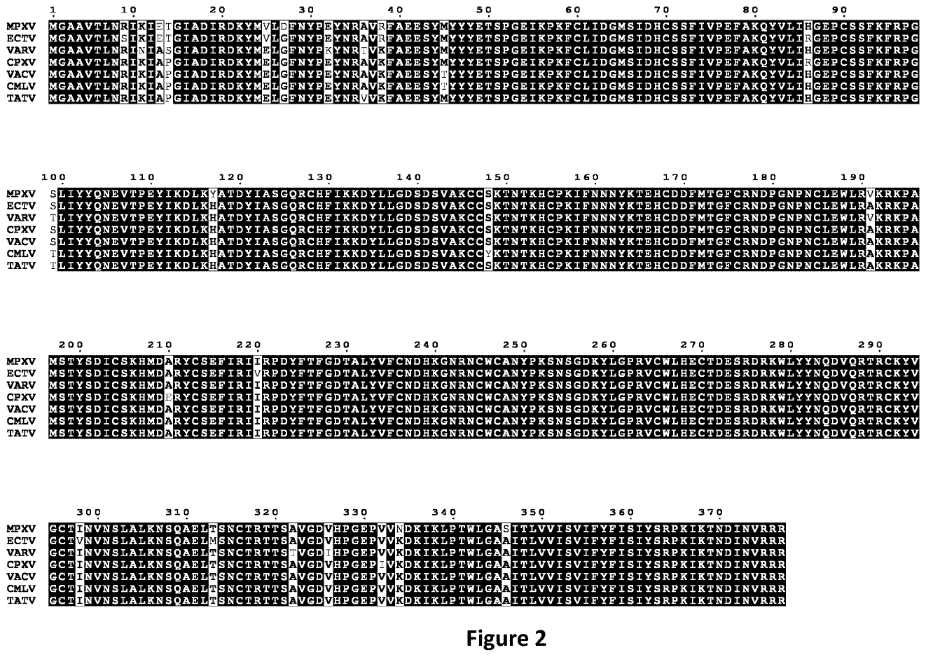

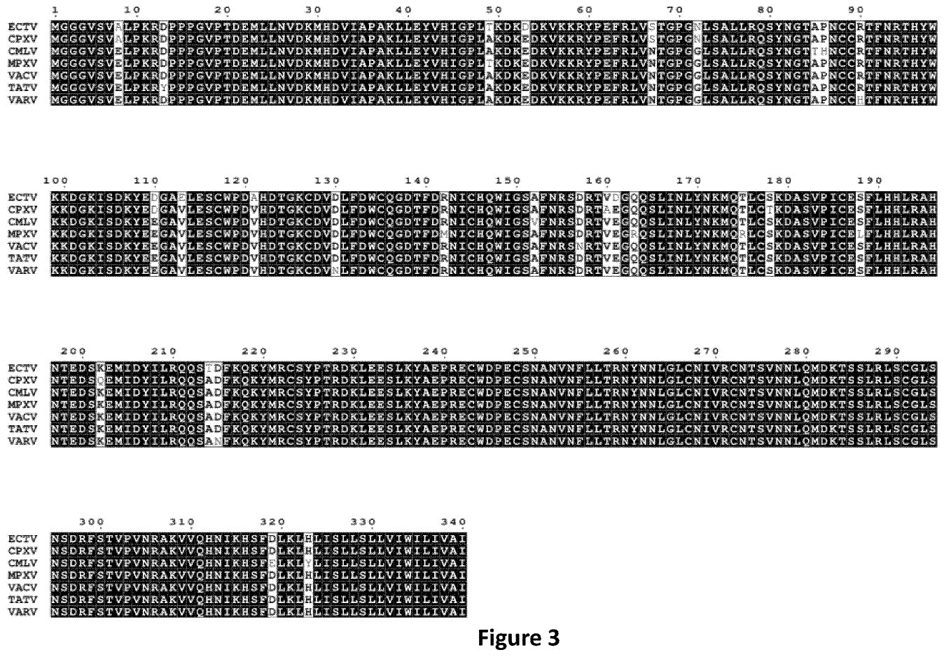

Two different heterodimers A16/G9 were produced using a bicistronic plasmid. A16 was tagged with a His-tag and G9 with a double strep-tag. In the first one, termed A16^{E}/G9^{E}, the full ectodomains (E) of A16 and G9 were cloned. In the second one, termed A16^{HB}/G9^{HB}, just the Head (H) and Base (B) domains were included. Based on the structural data (FIG. 1) and sequence alignments (FIGS. 2 and 3), the following mutations were introduced:

### Modifications in A16:

G2A to remove the myristoylation motif
T317A to remove the N-glycosylation in N315

### Modifications in G9:

G2A to remove the myristoylation motif
N82A to remove the N-glycosylation in N82
N93Q to remove the N-glycosylation in N93
S156A to remove the N-glycosylation in N154
N157D to remove the N-glycosylation in N157
S275N to remove the N-glycosylation in N273

The final sequences for both constructs are the following. The N-ter signal peptide and C-ter purification tags are underlined. The mutations are in bold.

A16-E
(SEQ ID NO: 15)
MKLCILLAVVAFVGLSLGRSAAAVTLNRIKIAPGIADIRDKYMELGFNYPEYNRAVKFAEESYTYYY
ETSPGEIKPKFCLIDGMSIDHCSSFIVPEFAKQYVLIHGEPCSSFKFRPGSLIYYQNEVTPEYIKDL
KHATDYIASGORCHFIKKDYLLGDSDSVAKCCSKTNTKHCPKIFNNNYKTEHCDDFMTGFCRNDPGN
PNCLEWLRAKRKPAMSTYSDICSKHMDARYCSEFIRIIRPDYFTFGDTALYVFCNDHKGNRNCWCAN
YPKSNSGDKYLGPRVCWLHECTDESRDRKWLYYNQDVQRTRCKYVGCTINVNSLALKNSQAELTSNC
ARTTSAVGDVHPGEPVVKDKIKGSGLVPRGSGGSGGSHHHHHHHHGGSGTGGLNDIFEAQKIEWHE*
G9-E
(SEQ ID NO: 16)
MKLCILLAVVAFVGLSLGAGGVSVELPKRDPPPGVPTDEMLLNVDKMHDVIAPAKLLEYVHIGPLAK
DKEDKVKKRYPEFRLVNTGPGGLSALLRQSYAGTAPNCCRTFQRTHYWKKDGKISDKYEEGAVLESC
WPDVHDTGKCDVDLEDWCQGDTFDRNICHQWIGSAFNRADRTVEGQQSLINLYNKMQTLCSKDASVP
ICESFLHHLRAHNTEDSKEMIDYILRQQSADFKQKYMRCSYPTRDKLEESLKYAEPRECWDPECSNA
NVNFLLTRNYNNLGLCNIVRCNTNVNNLQMDKTSSLRLSCGLSNSDRESTVPVNRAKVVQHNIKHSF
DLKGSGLVPRGSLEDDDDKAGWSHPQFEKGGGSGGGSGGGSWSHPQFEK*
A16-HB
(SEQ ID NO: 17)
MKLCILLAVVAFVGLSLGRSAAAVTLNRIKIAPGIADIRDKYMELGFNYPEYNRAVKFAEESYTYYY
ETSPGEIKPKFCLIDGMSIDHCSSFIVPEFAKQYVLIHGEPCSSFKFRPGSLIYYQNEVTPEYIKDL
KHATDYIASGQRCHFIKKDYLLGDSDSVAKCCSKTNTKHCPKIFNNNYKTEHCDDFMTGFCRNDPGN
PNCLEWLRAKRKPAMSTYSDICSKHMDARYCSEFIRIIRPDYFTFGDTALYVFCNDHKGNRNCWCAN
YPKSNSGDKYLGPRVCWLHECTDESRDRKWLYYNQDVQRTRCKYVGGSGLVPRGSGGSGGSHHHHHH
HHGGSGTGGLNDIFEAQKIEWHE*
G9-HB
(SEQ ID NO: 18)
MKLCILLAVVAFVGLSLGAGGVSVELPKRDPPPGVPTDEMLLNVDKMHDVIAPAKLLEYVHIGPLAK
DKEDKVKKRYPEFRLVNTGPGGLSALLRQSYAGTAPNCCRTFQRTHYWKKDGKISDKYEEGAVLESC
WPDVHDTGKCDVDLFDWCQGDTFDRNICHQWIGSAFNRADRTVEGQQSLINLYNKMQTLCSKDASVP
ICESFLHHLRAHNTEDSKEMIDYILRQQSADFKQKYMRCSYPTRDKLEESLKYAEPRECWDPECSNA
NVNFLLTRNYNNLGLCNIVRGSGLVPRGSLEDDDDKAGWSHPQFEKGGGSGGGSGGGSWSHPQFEK*

The coding sequences are as follows: A16-E (SEQ ID NO: 23); G9-E (SEQ ID NO: 24); A16-HB (SEQ ID NO: 25); G9-HB (SEQ ID NO: 26).

### Protein Expression, Purification and Biochemical Characterization

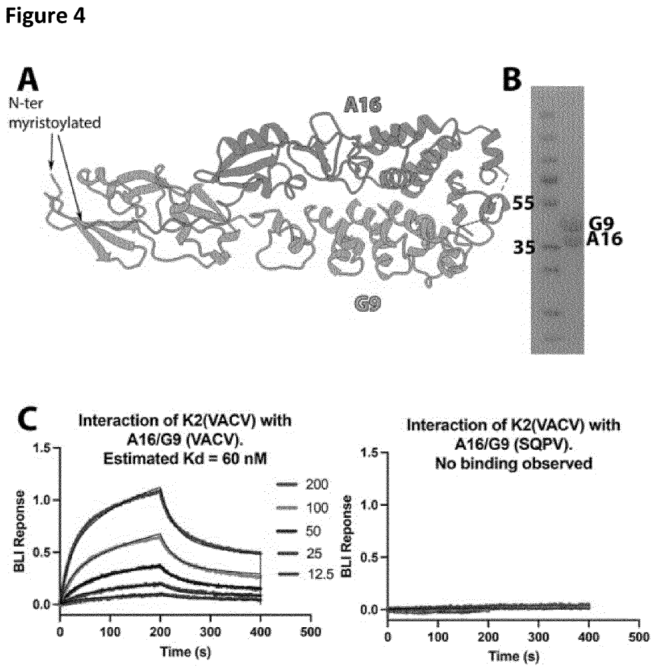

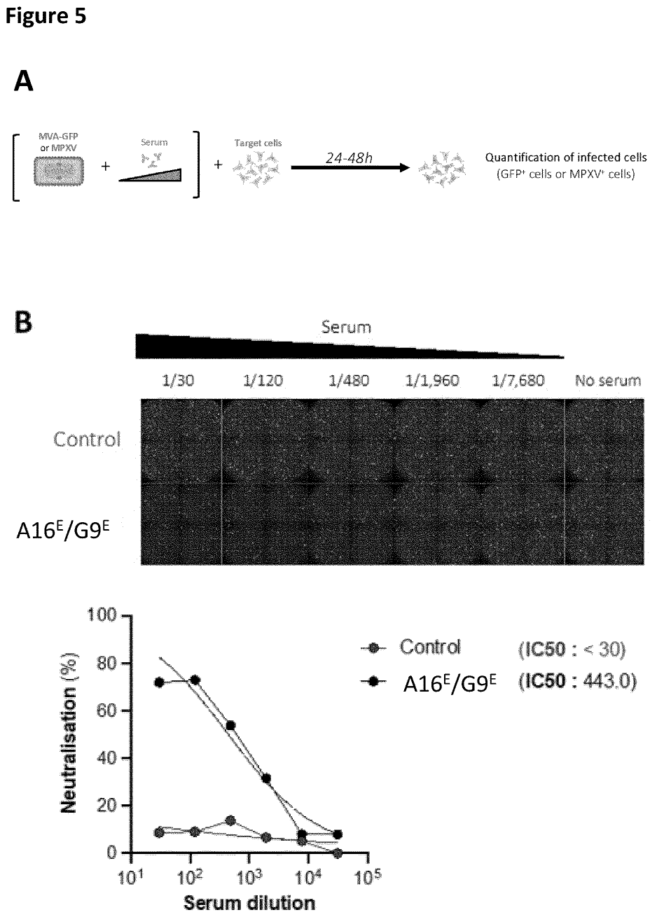

Both constructs were produced using insect cells (Drosophila melanogaster S2 cells). Both constructs are produced in large quantities. The complex was purified using two affinity chromatographies. The first one using streptactin columns, which recognise the G9 streptag, and then Ni-NTA, which recognises the A16 His-tag. A size-exclusion chromatography was then performed, which yielded a single peak compatible with the size of a heterodimer and confirmed the presence of both proteins by western blot using specific antibodies against G9 and A16. SDS-PAGE analysis shows that A16^{E}/G9^{E} forms disulphide bonds crosslinking both subunits, which is believed to be an artefact of purification, i.e. both subunits are not cross-linked on the viral surface. The complex A16^{E}/G9^{E} from squirrelpox virus, a member of the genus sciuripoxvirus was also expressed and purified using exactly the same strategy described above. To demonstrate that the heterodimer folds as on the viral surface, the complex A16^{E}/G9^{E} from squirrelpox virus, was crystallised (FIG. 4A), which has been expressed and purified using exactly the same strategy described above. As can be seen, the crystal structure of the complex closely resembles the model of the complex predicted by alphafold for vaccinia virus (FIG. 1). Binding experiments were also performed using one of the fusion suppressors (A56/K2), which targets the viral A16/G9 and blocks fusion. The BLI experiments showed that complex A56/K2 of vaccinia virus interacts specifically with the subcomplex A16^{HB}/G9^{HB} of vaccinia virus (FIG. 4C).

### Immunization of Animals, Isolation of Neutralizing Monoclonal Antibodies and Neutralization Experiments

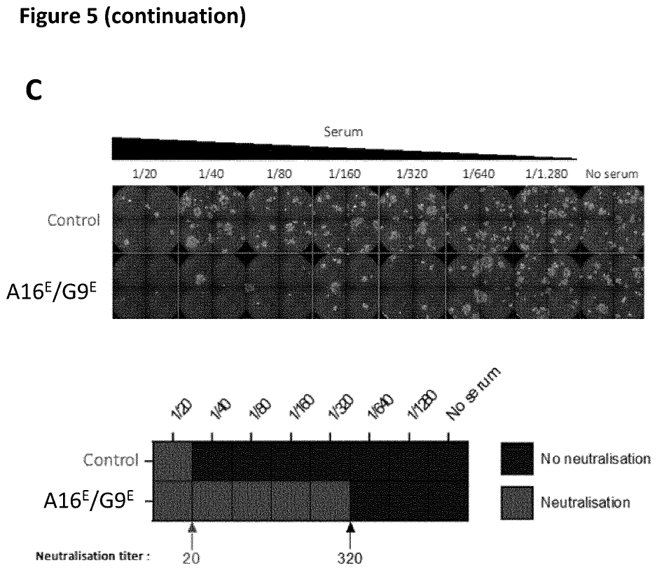

To prove that these constructs produce neutralizing antibodies and can be used as immunogens, A16^{E}/G9^{E} was used to immunise one alpaca. The ability of the serum to neutralize MVA (Modified vaccinia virus) and monkeypox was then determined using a microneutralization assay (FIG. 5). In these experiments, serum from an alpaca that has been immunised with an unrelated protein was used as a negative control. The output of the experiments is a ED50 value for MVA, which is the dilution of serum necessary to reduce MVA-GFP infection by 50%, and a neutralization titer for MPXV, which the highest dilution in which neutralization was observed. As can be seen in the experiment, serum from alpaca immunised with the engineered heterodimer neutralises MVA with an ED50=443 and MPXV with a neutralization titer of 320. These results confirm that the heterodimer thus designed and purified is able to induce neutralizing antibodies against both viruses.

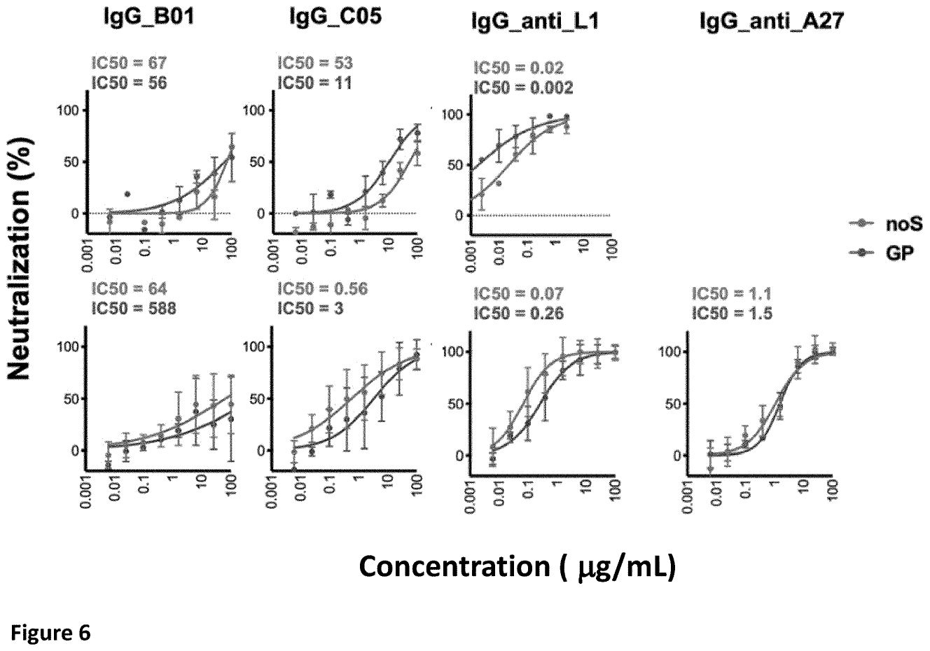

Monoclonal antibodies (Ig-VHH) directed against the heterodimer A16^{E}/G9^{E} having a neutralizing activity in the absence of complement were isolated (FIG. 6). Their neutralizing power is similar to that of the best neutralizing antibody previously isolated against A27. They neutralize at least two different orthopoxvirus: Vaccinia virus (VACV) and Monkeypoxvirus (MPXV).

TABLE 2
Sequences disclosed in the present application

| SEQ | | | |
|---|---|---|---|
| ID | | | |
| NO: | Name | Type | Sequence |
| 1 | VACV-A16 | PRT | MGAAVTLNRI KIAPGIADIR DKYMELGFNY PEYNRAVKFA |
| | | | EESYTYYYET SPGEIKPKFC LIDGMSIDHC SSFIVPEFAK |
| | | | QYVLIHGEPC SSFKFRPGSL IYYQNEVTPE YIKDLKHATD |
| | | | YIASGQRCHF IKKDYLLGDS DSVAKCCSKT NTKHCPKIFN |
| | | | NNYKTEHCDD FMTGFCRNDP GNPNCLEWLR AKRKPAMSTY |
| | | | SDICSKHMDA RYCSEFIRII RPDYFTFGDT ALYVFCNDHK |
| | | | GNRNCWCANY PKSNSGDKYL GPRVCWLHEC TDESRDRKWL |
| | | | YYNQDVQRTR CKYVGCTINV NSLALKNSQA ELTSNCTRTT |
| | | | SAVGDVHPGE PVVKDKIKLP TWLGAAITLV VISVIFYFIS |
| | | | IYSRPKIKTN DINVRRR |
| 2 | CMLV-A16 | PRT | MGAAVTLNRI KIAPGIADIR DKYMELGFNY PEYNRAVKFA |
| | | | EESYTYYYET SPGEIKPKFC LIDGMSIDHC SSFIVPEFAK |
| | | | QYVLIHGEPC SSFKFRPGTL IYYQNEVTPE YIKDLKHATD |
| | | | YIASGQRCHF IKKDYLLGDS DSVAKCCYKT NTKHCPKIFN |
| | | | NNYKTEHCDD FMTGFCRNDP GNPNCLEWLR AKRKPAMSTY |
| | | | SDICSKHMDA RYCSEFIRII RPDYFTFGDT ALYVFCNDHK |
| | | | GNRNCWCANY PKSNSGDKYL GPRVCWLHEC TDESRDRKWL |
| | | | YYNQDVQRTR CKYVGCTINV NSLALKNSQA ELTSNCTRTT |
| | | | SAVGDVHPGE PVVKDKIKLP TWLGAAITLV VISVIFYFIS |
| | | | IYSRPKIKTN DINVRRR |
| 3 | TATV-A16 | PRT | MGAAVTLNRI KIAPGIADIR DKYMELGFNY PEYNRVVKFA |
| | | | EESYMYYYET SPGEIKPKFC LIDGMSIDHC SSFIVPEFAK |
| | | | QYVLIHGEPC SSFKFRPGTL IYYQNEVTPE YIKDLKHATD |
| | | | YIASGQRCHF IKKDYLLGDS DSVAKCCSKT NTKHCPKIFN |
| | | | NNYKTEHCDD FMTGFCRNDP GNPNCLEWLR AKRKPAMSTY |
| | | | SDICSKHMDA RYCSEFIRII RPDYFTFGDT ALYVFCNDHK |
| | | | GNRNCWCANY PKSNSGDKYL GPRVCWLHEC TDESRDRKWL |
| | | | YYNQDVQRTR CKYVGCTINV NSLALKNSQA ELTSNCTRTT |
| | | | SAVGDVHPGE PVVKDKIKLP TWLGAAITLV VISVIFYFIS |
| | | | IYSRPKIKTN DINVRRR |
| 4 | CPXV-A16 | PRT | MGAAVTLNRI KIAPGIADIR DKYMELGFNY PEYNRAVKFA |
| | | | EESYMYYYET SPGEIKPKFC LIDGMSIDHC SSFIVPEFAK |
| | | | QYVLIRGEPC SSFKFRPGSL IYYQNEVTPE YIKDLKHATD |
| | | | YIASGQRCHF IKKDYLLGDS DSVAKCCSKT NTKHCPKIFN |
| | | | NNYKTEHCDD FMTGFCRNDP GNPNCLEWLR AKRKPAMSTY |
| | | | SDICSKHMDE RYCSEFIRII RPDYFTFGDT ALYVFCNDHK |
| | | | GNRNCWCANY PKSNSGDKYL GPRVCWLHEC TDESRDRKWL |
| | | | YYNQDVQRTR CKYVGCTINV NSLALKNSQA ELTSNCTRTT |
| | | | SAVGDVHPGE PIVKDKIKLP TWLGAAITLV VISVIFYFIS |
| | | | IYSRPKIKTN DINVRRR |
| 5 | VARV-A16 | PRT | MGAAVTLNRI NIASGIADIR DKYMELGFNY PKYNRTVKFA |
| | | | EESYMYYYET SPGEIKPKFC LIDGMSIDHC SSFIVPEFAK |
| | | | QYVLINGEPC SSFKFRPGTL IYYQNEVTPE YIKDLKHATD |
| | | | YIASGQRCHF IKKDYLLGDS DSVAKCCSKT NTKHCPKIFN |
| | | | NNYKTEHCDD FMTGFCRNDP GNPNCLEWLR VKRKPAMSTY |
| | | | SDICSKHMDA RYCSEFIRII RPDYFTFGDT ALYVFCNDHK |
| | | | GNRNCWCANY PKSNSGDKYL GPRVCWLHEC TDESRDRKWL |
| | | | YYNQDVQRTR CKYVGCTINV NSLALKNSQA ELTSNCTRTT |
| | | | STVGDIHPGE PVVKDKIKLP TWLGAAITLV VISVIFYFIS |
| | | | IYSRPKIKTN DINVRRR |
| 6 | ECTV-A16 | PRT | MGAAVTLNSI KIETGIADIR DKYMVLGFNY PEYNRAVRFA |
| | | | EESYMYYYET SPGEIKPKFC LIDGMSIDHC SSFIVPEFAK |
| | | | QYVLIRGEPC SSFKFRPGSL IYYQNEVTPE YIKDLKHATD |
| | | | YIASGQRCHF IKKDYLLGDS DSVAKCCSKT NTKHCPKIFN |
| | | | NNYKTEHCDD FMTGFCRNDP GNPNCLEWLR AKRKPAMSTY |
| | | | SDICSKHMDA RYCSEFIRIV RPDYFTFGDT ALYVFCNDHK |
| | | | GNRNCWCANY PKSNSGDKYL GPRVCWLHEC TDESRDRKWL |
| | | | YYNQDVQRTR CKYVGCTVNV NSLALKNSQA ELMSNCTRTT |
| | | | SAVGDVHPGE PVVKDKIKLP TWLGAAITLV VISVIFYFIS |
| | | | IYSRPKIKTN DINVRRR |
| 7 | MPXV-A16 | PRT | MGAAVTLNRI KIETGIADIR DKYMVLDFNY PEYNRAVRFA |
| | | | EESYMYYYET SPGEIKPKFC LIDGMSIDHC SSFIVPEFAK |
| | | | QYVLIHGEPC SSFKFRPGSL IYYQNEVTPE YIKDLKYATD |
| | | | YIASGQRCHF IKKDYLLGDS DSVAKCCSKT NTKHCPKIFN |
| | | | NNYKTEHCDD FMTGFCRNDP GNPNCLEWLR VKRKPAMSTY |
| | | | SDICSKHMDA RYCSEFIRII RPDYFTFGDT ALYVFCNDHK |
| | | | GNRNCWCANY PKSNSGDKYL GPRVCWLHEC TDESRDRKWL |
| | | | YYNQDVQRTR CKYVGCTINV NSLALKNSQA ELTSNCTRTT |
| | | | SAVGDVHPGE PVVNDKIKLP TWLGASITLV VISVIFYFIS |
| | | | IYSRPKIKTN DINVRRR |
| 8 | VACV-G9 | PRT | MGGGVSVELP KRDPPPGVPT DEMLLNVDKM HDVIAPAKLL |
| | | | EYVHIGPLAK DKEDKVKKRY PEFRLVNTGP GGLSALLRQS |
| | | | YNGTAPNCCR TFNRTHYWKK DGKISDKYEE GAVLESCWPD |
| | | | VHDTGKCDVD LFDWCQGDTF DRNICHQWIG SAFNRSNRTV |
| | | | EGQQSLINLY NKMQTLCSKD ASVPICESFL HHLRAHNTED |
| | | | SKEMIDYILR QQSADFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFDL |
| | | | KLHLISLLSL LVIWILIVAI |
| 9 | TATV-G9 | PRT | MGGGVSVELP KRYPPPGVPT DEMLLNVDKM HDVIAPAKLL |
| | | | EYVHIGPLAK DKEDKVKKRY PEFRLVNTGP GGLSALLRQS |
| | | | YNGTAPNCCR TFNRTHYWKK DGKISDKYEE GAVLESCWPD |
| | | | VHDTGKCDVD LFDWCQGDTF DRNICHQWIG SAFNRSDRTV |
| | | | EGQQSLINLY NKMQTLCSKD ASVPICESFL HHLRAHNTED |
| | | | SKEMIDYILR QQSADFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFDL |
| | | | KLHLISLLSL LVIWILIVAI |
| 10 | VARV-G9 | PRT | MGGGVSVELP KRDPPPGVPT DEMLLNVDKM HDVIAPAKLL |
| | | | EYVHIGPLAK DKEDKVKKRY PEFRLVNTGP GGLSALLRQS |
| | | | YNGTAPNCCH TFNRTHYWKK DGKISDKYEE GAVLESCWPD |
| | | | VHDTGKCDVN LEDWCQGDTF DRNICHQWIG SAFNRSDRTV |
| | | | EGQQSLINLY NKMQTLCSKD ASVPICESFL HHLRAHNTED |
| | | | SKEMIDYILR QQSANFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFDL |
| | | | KLHLISLLSL LVIWILIVAI |
| 11 | MPXV-G9 | PRT | MGGGVSVELP KRDPPPGVPT DEMLLNVDKM HDVIAPAKLL |
| | | | EYVHIGPLTK DKEDKVKKRY PEFRLVNTGP GGLSALLRQS |
| | | | YNGTAPNCCR TFNRTHYWKK DGKISDKYEE GAVLESCWPD |
| | | | VHDTGKCDVD LFDWCQGDTF DMNICHQWIG SAFNRSDRTV |
| | | | EGRQSLINLY NKMQRLCSKD ASVPICELFL HHLRAHNTED |
| | | | SKEMIDYILR QQSADFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFDL |
| | | | KLHLISLLSL LVIWILIVAI |
| 12 | CMLV-G9 | PRT | MGGGVSVELP KRDPPPGVPT DEMLLNVDKM HDVIAPAKLL |
| | | | EYVHIGPLAK DKEDKVKKRY PEFRLVNTGP GGLSALLRQS |
| | | | YNGTTHNCCR TFNRTHYWKK DGKISDKYEE GAVLESCWPD |
| | | | VHDTGKCDVD LEDWCQGDTF DRNICHQWIG SVFNRSDRTV |
| | | | EGQQSLINLY NKMQTLCSKD ASVPICESFL HHLRAHNTED |
| | | | SKEMIDYILR QQSADFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFEL |
| | | | KLYLISLLSL LVIWILIVAI |
| 13 | CPXV-G9 | PRT | MGGGVSVALP KRDPPPGVPT DEMLLNVDKM HDVIAPAKLL |
| | | | EYVHIGPLAK DKEDKVKKRY PEFRLVSTGP GNLSALLRQS |
| | | | YNGTAPNCCR TFNRTHYWKK DGKISDKYED GAVLESCWPD |
| | | | VHDTGKCDVD LEDWCQGDTF DRNICHQWIG SAFNRSDRTA |
| | | | EGQQSLINLY NKMQTLCTKD ASVPICESFL HHLRAHNTED |
| | | | SQEMIDYILR QQSADFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFDL |
| | | | KLHLISLLSL LVIWILIVAI |
| 14 | ECTV-G9 | PRT | MGGGVSVALP KRDPPPGVPT DEMLLNVDKM HDVIAPAKLL |
| | | | EYVHIGPLTK DKDDKVKKRY PEFRLVSTGP GNLSALLRQS |
| | | | YNGTAPNCCR TFNRTHYWKK DGKISDKYED GAELESCWPD |
| | | | AHDTGKCDVD LFDWCQGDTF DRNICHQWIG SAFNRSDRTV |
| | | | DGQQSLINLY NKMQTLCSKD ASVPICESFL HHLRAHNTED |
| | | | SKEMIDYILR QQSTDFKQKY MRCSYPTRDK LEESLKYAEP |
| | | | RECWDPECSN ANVNFLLTRN YNNLGLCNIV RCNTSVNNLQ |
| | | | MDKTSSLRLS CGLSNSDRFS TVPVNRAKVV QHNIKHSFDL |
| | | | KLHLISLLSL LVIWILIVAI |
| 15 | A16-E | PRT | MKLCILLAVVAFVGLSLGRSAAAVTLNRIKIAPGIADIRDKYMEL |
| | | | GFNYPEYNRAVKFAEESYTYYYETSPGEIKPKFCLIDGMSIDHCS |
| | | | SFIVPEFAKQYVLIHGEPCSSFKFRPGSLIYYQNEVTPEYIKDLK |
| | | | HATDYIASGQRCHFIKKDYLLGDSDSVAKCCSKTNTKHCPKIFNN |
| | | | NYKTEHCDDFMTGFCRNDPGNPNCLEWLRAKRKPAMSTYSDICSK |
| | | | HMDARYCSEFIRIIRPDYFTFGDTALYVFCNDHKGNRNCWCANYP |
| | | | KSNSGDKYLGPRVCWLHECTDESRDRKWLYYNQDVQRTRCKYVGC |
| | | | TINVNSLALKNSQAELTSNCARTTSAVGDVHPGEPVVKDKIKGSG |
| | | | LVPRGSGGSGGSHHHHHHHHGGSGTGGLNDIFEAQKIEWHE |
| 16 | G9-E | PRT | MKLCILLAVVAFVGLSLGAGGVSVELPKRDPPPGVPTDEMLLNVD |
| | | | KMHDVIAPAKLLEYVHIGPLAKDKEDKVKKRYPEFRLVNTGPGGL |
| | | | SALLRQSYAGTAPNCCRTFQRTHYWKKDGKISDKYEEGAVLESCW |
| | | | PDVHDTGKCDVDLFDWCQGDTFDRNICHQWIGSAFNRADRTVEGQ |
| | | | QSLINLYNKMQTLCSKDASVPICESFLHHLRAHNTEDSKEMIDYI |
| | | | LRQQSADFKQKYMRCSYPTRDKLEESLKYAEPRECWDPECSNANV |
| | | | NFLLTRNYNNLGLCNIVRCNTNVNNLQMDKTSSLRLSCGLSNSDR |
| | | | FSTVPVNRAKVVQHNIKHSFDLKGSGLVPRGSLEDDDDKAGWSHP |
| | | | QFEKGGGSGGGSGGGSWSHPQFEK |
| 17 | A16-HB | PRT | MKLCILLAVVAFVGLSLGRSAAAVTLNRIKIAPGIADIRDKYMEL |
| | | | GFNYPEYNRAVKFAEESYTYYYETSPGEIKPKFCLIDGMSIDHCS |
| | | | SFIVPEFAKQYVLIHGEPCSSFKFRPGSLIYYQNEVTPEYIKDLK |
| | | | HATDYIASGQRCHFIKKDYLLGDSDSVAKCCSKTNTKHCPKIFNN |
| | | | NYKTEHCDDFMTGFCRNDPGNPNCLEWLRAKRKPAMSTYSDICSK |
| | | | HMDARYCSEFIRIIRPDYFTFGDTALYVFCNDHKGNRNCWCANYP |
| | | | KSNSGDKYLGPRVCWLHECTDESRDRKWLYYNQDVQRTRCKYVGG |
| | | | SGLVPRGSGGSGGSHHHHHHHHGGSGTGGLNDIFEAQKIEWHE |
| 18 | G9-HB | PRT | MKLCILLAVVAFVGLSLGAGGVSVELPKRDPPPGVPTDEMLLNVD |
| | | | KMHDVIAPAKLLEYVHIGPLAKDKEDKVKKRYPEFRLVNTGPGGL |
| | | | SALLRQSYAGTAPNCCRTFQRTHYWKKDGKISDKYEEGAVLESCW |
| | | | PDVHDTGKCDVDLFDWCQGDTFDRNICHQWIGSAFNRADRTVEGQ |
| | | | QSLINLYNKMQTLCSKDASVPICESFLHHLRAHNTEDSKEMIDYI |
| | | | LRQQSADFKQKYMRCSYPTRDKLEESLKYAEPRECWDPECSNANV |
| | | | NFLLTRNYNNLGLCNIVRGSGLVPRGSLEDDDDKAGWSHPQFEKG |
| | | | GGSGGGSGGGSWSHPQFEK |
| 19 | Signal | PRT | MKLCILLAVVAFVGLSLG |
| | peptide of D. | | |
| | melanogaster | | |
| | Hsc70-3 (BiP) | | |
| 20 | His-tag | PRT | GSGLVPRGSGGSGGSHHHHHHHHGGSGTGGLNDIFEAQKIEWHE |
| 21 | double | PRT | GSGLVPRGSLEDDDDKAGWSHPQFEKGGGSGGGSGGGSWSHPQFE |
| | strep-tag | | K |
| 22 | Signal | | MYRMQLLSCIALSLALVINS |
| | peptide of | | |
| | IL-2 | | |
| 23 | A16-E | DNA | atgaagttatgcatattactggccgtcgtggcctttgttggcctc |
| | | | tcgctcgggagatctgccgccgccgtgaccctgaaccgcatcaag |
| | | | atcgccccaggcatcgccgatatccgcgacaagtacatggagctg |
| | | | ggcttcaactaccccgagtacaatcgcgccgtgaagttcgccgag |
| | | | gagagctacacctactactacgagacctcccccggcgagatcaag |
| | | | ccgaagttctgcctgatcgatggcatgtcgatcgaccactgcagc |
| | | | tccttcatcgtgccggagttcgccaagcagtacgtgctgatccac |
| | | | ggcgagccgtgctcgagcttcaagttccgcccaggcagcctgatc |
| | | | tactaccagaacgaggtgaccccagagtacatcaaggatctgaag |
| | | | cacgccaccgactacattgcctcgggacagcgctgccacttcatc |
| | | | aagaaggattacctgctgggcgatagcgactccgtggccaagtgc |
| | | | tgctccaagaccaacaccaagcactgccccaagattttcaacaat |
| | | | aactacaagaccgagcactgcgatgacttcatgaccggcttctgc |
| | | | cgcaatgacccaggcaatcccaactgcctggagtggctgcgcgcc |
| | | | aagcgcaagccagccatgtccacctacagcgatatctgctccaag |
| | | | cacatggacgcccgctactgctcggagttcatccgcatcatccgc |
| | | | ccggattacttcaccttcggcgacaccgccctgtacgtgttctgc |
| | | | aacgatcacaagggcaatcgcaactgctggtgcgccaactacccg |
| | | | aagtcgaatagcggcgacaagtacctgggaccccgcgtgtgctgg |
| | | | ctgcacgagtgcaccgatgagtcccgcgaccgcaagtggctgtac |
| | | | tacaatcaggatgtgcagcgcacccgctgcaagtacgtgggctgc |
| | | | accatcaatgtgaacagcctggccctgaagaactcccaggccgag |
| | | | ctgacctccaattgcgcccgcaccacctcggccgtgggcgatgtg |
| | | | cacccgggcgagccagtggtgaaggacaagatcaagggcagcggc |
| | | | ctggtgccacgcattggaagcggcggctccggcggctcgcaccat |
| | | | catcaccaccaccaccacggcggctcgggaaccggcggcctgaac |
| | | | gatatcttcgaggcccagaagatcgagtggcacgagtaa |
| 24 | G9-E | DNA | atgaagttatgcatattactggccgtcgtggcctttgttggcctc |
| | | | agcctaggagccggcggcgtgtcggtggagctgccgaagcgcgat |
| | | | cccccgccaggagtgccgaccgacgagatgctgctgaacgtggat |
| | | | aagatgcacgacgtgatcgcccccgccaagctgctggagtacgtg |
| | | | cacatcggcccactggccaaggataaggaggacaaggtgaagaag |
| | | | cgctacccagagttccgcctggtgaacaccggccccggcggcctg |
| | | | tcggccctgctgcgccagtcctacgccggaaccgcccccaattgc |
| | | | tgccgcaccttccagcgcacccactactggaagaaggatggcaag |
| | | | atcagcgacaagtacgaggagggagccgtgctggagtcctgctgg |
| | | | cccgatgtgcacgacaccggcaagtgcgatgtggacctgttcgat |
| | | | tggtgccagggcgataccttcgaccgcaacatctgccaccagtgg |
| | | | atcggctcggccttcaatcgcgccgatcgcaccgtggagggacag |
| | | | cagagcctgatcaacctgtacaataagatgcagaccctgtgctcc |
| | | | aaggacgcctcggtgccgatctgcgagtccttcctgcaccacctg |
| | | | cgcgcccacaacaccgaggatagcaaggagatgatcgactacatc |
| | | | ctgcgccagcagtccgccgatttcaagcagaagtacatgcgctgc |
| | | | agctaccccacccgcgacaagctggaggagtccctgaagtacgcc |
| | | | gagccacgcgagtgctgggatccagagtgcagcaatgccaacgtg |
| | | | aatttcctgctgacccgcaactacaataacctgggcctgtgcaat |
| | | | atcgtgcgctgcaacaccaatgtgaataacctgcagatggataag |
| | | | acctcctccctgcgcctgtcctgcggactgagcaactccgatcgc |
| | | | ttctcgaccgtgcccgtgaaccgcgccaaggtggtgcagcacaat |
| | | | atcaagcactcgttcgacctgaagggatcgggcctggtgccccgc |
| | | | atcggctccctcgaggacgatgacgataaggccggttggagtcat |
| | | | ccacaattcgagaagggcggcggctccggaggtggatcaggaggt |
| | | | ggttcctggtcacaccctcaattcgagaagtga |
| 25 | A16-HB | DNA | atgaagttatgcatattactggccgtcgtggcctttgttggcctc |
| | | | tcgctcgggagatctgccgccgccgtgaccctgaaccgcatcaag |
| | | | atcgccccaggcatcgccgatatccgcgacaagtacatggagctg |
| | | | ggcttcaactaccccgagtacaatcgcgccgtgaagttcgccgag |
| | | | gagagctacacctactactacgagacctcccccggcgagatcaag |
| | | | ccgaagttctgcctgatcgatggcatgtcgatcgaccactgcagc |
| | | | tccttcatcgtgccggagttcgccaagcagtacgtgctgatccac |
| | | | ggcgagccgtgctcgagcttcaagttccgcccaggcagcctgatc |
| | | | tactaccagaacgaggtgaccccagagtacatcaaggatctgaag |
| | | | cacgccaccgactacattgcctcgggacagcgctgccacttcatc |
| | | | aagaaggattacctgctgggcgatagcgactccgtggccaagtgc |
| | | | tgctccaagaccaacaccaagcactgccccaagattttcaacaat |
| | | | aactacaagaccgagcactgcgatgacttcatgaccggcttctgc |
| | | | cgcaatgacccaggcaatcccaactgcctggagtggctgcgcgcc |
| | | | aagcgcaagccagccatgtccacctacagcgatatctgctccaag |
| | | | cacatggacgcccgctactgctcggagttcatccgcatcatccgc |
| | | | ccggattacttcaccttcggcgacaccgccctgtacgtgttctgc |
| | | | aacgatcacaagggcaatcgcaactgctggtgcgccaactacccg |
| | | | aagtcgaatagcggcgacaagtacctgggaccccgcgtgtgctgg |
| | | | ctgcacgagtgcaccgatgagtcccgcgaccgcaagtggctgtac |
| | | | tacaatcaggatgtgcagcgcacccgctgcaagtacgtgggcggc |
| | | | agcggcctggtgccacgcggaagcggcggctccggcggctcgcac |
| | | | catcatcaccaccaccaccacggcggctcgggaaccggcggcctg |
| | | | aacgatatcttcgaggcccagaagatcgagtggcacgagtaa |
| 26 | G9-HB | DNA | atgaagttatgcatattactggccgtcgtggcctttgttggcctc |
| | | | agcctaggagccggcggcgtgtcggtggagctgccgaagcgcgat |
| | | | cccccgccaggagtgccgaccgacgagatgctgctgaacgtggat |
| | | | aagatgcacgacgtgatcgcccccgccaagctgctggagtacgtg |
| | | | cacatcggcccactggccaaggataaggaggacaaggtgaagaag |
| | | | cgctacccagagttccgcctggtgaacaccggccccggcggcctg |
| | | | tcggccctgctgcgccagtcctacgccggaaccgcccccaattgc |
| | | | tgccgcaccttccagcgcacccactactggaagaaggatggcaag |
| | | | atcagcgacaagtacgaggagggagccgtgctggagtcctgctgg |
| | | | cccgatgtgcacgacaccggcaagtgcgatgtggacctgttcgat |
| | | | tggtgccagggcgataccttcgaccgcaacatctgccaccagtgg |
| | | | atcggctcggccttcaatcgcgccgatcgcaccgtggagggacag |
| | | | cagagcctgatcaacctgtacaataagatgcagaccctgtgctcc |
| | | | aaggacgcctcggtgccgatctgcgagtccttcctgcaccacctg |
| | | | cgcgcccacaacaccgaggatagcaaggagatgatcgactacatc |
| | | | ctgcgccagcagtccgccgatttcaagcagaagtacatgcgctgc |
| | | | agctaccccacccgcgacaagctggaggagtccctgaagtacgcc |
| | | | gagccacgcgagtgctgggatccagagtgcagcaatgccaacgtg |
| | | | aatttcctgctgacccgcaactacaataacctgggcctgtgcaat |
| | | | atcgtgcgcggatcgggcctggtgccccgcggctccctcgaggac |
| | | | gatgacgataaggccggttggagtcatccacaattcgagaagggc |
| | | | ggcggctccggaggtggatcaggaggtggttcctggtcacaccct |
| | | | caattcgagaagtga |

1-16. (canceled)

17. A method for immunizing a subject against a poxvirus infection or related disease, comprising administering to the subject an isolated heterodimer of poxvirus A16 and G9 proteins or a nucleic acid encoding said heterodimer.

18. The method of claim 17, wherein neutralizing antibodies against the poxvirus are induced in the subject following administration of the heterodimer.

19. The method of claim 17, wherein the A16 and G9 proteins are from orthopoxvirus chosen from: Vaccinia virus, Monkeypox virus, Ectromelia virus, Variola virus, Cowpox virus, Camelpox virus, and Tatera poxvirus.

20. The method of claim 17, wherein the heterodimer comprises truncated A16 and G9 proteins comprising the ectodomain or a fragment thereof comprising the beta-folded head domain and the small alpha helices rich domain.

21. The method of claim 17, wherein the heterodimer comprises truncated A16 and G9 proteins comprising an amino acid sequence selected from the group consisting of:
a sequence having at least 85% identity with the sequence from position 1 to any one of positions 333 to 343 of SEQ ID NO: 1; preferably having at least 85% identity with the sequence from position 1 to position 338 of SEQ ID NO: 1;
a sequence having at least 85% identity with the sequence from position 1 to any one of positions 287 to 297 of SEQ ID NO: 1; preferably having at least 85% identity with the sequence from position 1 to position 295 of SEQ ID NO: 1;
a sequence having at least 85% identity with the sequence from position 1 to any one of positions 316 to 326 of SEQ ID NO: 8; preferably having at least 85% identity with the sequence from position 1 to position 321 of SEQ ID NO: 1; and
a sequence having at least 85% identity with the sequence from position 1 to any one of positions 263 to 273 of SEQ ID NO: 8; preferably having at least 85% identity with the sequence from position 1 to position 271 of SEQ ID NO: 1.

22. The method of claim 17, wherein the A16 and G9 proteins are produced from a precursor comprising a N-terminal signal peptide.

23. The method of claim 17, wherein the A16 and G9 proteins comprise the G2A substitution.

24. The method of claim 17, wherein the A16 protein comprises the T317A substitution and the G9 protein comprises one or more or all of the substitutions N82A, N93Q, S156A, N157D and S275N, said positions being determined by alignment with A16 of SEQ ID NO: 1 and G9 of SEQ ID NO: 8.

25. The method of claim 17, wherein the truncated A16 and G9 proteins comprise an amino acid sequence chosen from any one of SEQ ID NO: 15 to 18.

26. The method of claim 17, wherein the A16 and G9 proteins are recombinant proteins.

27. The method of claim 17, wherein the nucleic acid is mRNA.

28. The method of claim 17, wherein the nucleic acid is inserted into a viral vector selected from the group consisting of: adenovirus, adeno-associated virus, vesicular stomatitis virus, lentivirus, poxvirus, vaccinia virus, modified vaccinia virus Ankara, insect-specific viral vectors, and combinations thereof.

29. The method of claim 1, which is for preventing or treating a Monkeypox virus infection or related disease in the subject.

30. An engineered heterodimer of poxvirus A16 and G9 proteins or a nucleic acid encoding said heterodimer, wherein the heterodimer comprises truncated A16 and G9 comprising the ectodomain or a fragment thereof comprising the beta-folded head domain and the small alpha helices rich domain as defined in claim 4.

31. A method for detecting the presence or absence of antibodies against poxvirus in a sample, comprising contacting the sample with the isolated heterodimer of poxvirus A16 and G9 proteins of claim 17 to form an immunoreaction product, and detecting the presence of the immunoreaction product.

32. A neutralizing monoclonal antibody against poxvirus, which is directed against the isolated heterodimer of poxvirus A16 and G9 proteins of claim 17.

33. The method of claim 27, wherein the mRNA is modified mRNA which is enclosed in a lipid nanoparticle (LNP).

34. The method of claim 17, which is for vaccinating the subject against a poxvirus infection or related disease in the subject.

35. The method of claim 17, which is for vaccinating the subject against a Monkeypox virus infection or related disease in the subject.

36. The method of claim 17, which is for preventing or treating a poxvirus infection or related disease in the subject.


SEQUENCE LISTING (26 sequences)

SEQ ID NO: 1 - protein, 377 aa, Vaccinia virus
MGAAVTLNRIKIAPGIADIRDKYMELGFNYPEYNRAVKFAEESYTYYYETSPGEIKPKFCLIDGMSIDHCSSFIVPEFAKQYVLIHGEPCSSFKFRPGSLIYYQNEVTPEYIKDLKHATDYIASGQRCHFIKKDYLLGDSDSVAKCCSKTNTKHCPKIFNNNYKTEHCDDFMTGFCRNDPGNPNCLEWLRAKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCTRTTSAVGDVHPGEPVVKDKIKLPTWLGAAITLVVISVIFYFISIYSRPKIKTNDINVRRR

SEQ ID NO: 2 - protein, 377 aa, Camelpox virus
MGAAVTLNRIKIAPGIADIRDKYMELGFNYPEYNRAVKFAEESYTYYYETSPGEIKPKFCLIDGMSIDHCSSFIVPEFAKQYVLIHGEPCSSFKFRPGTLIYYQNEVTPEYIKDLKHATDYIASGQRCHFIKKDYLLGDSDSVAKCCYKTNTKHCPKIFNNNYKTEHCDDFMTGFCRNDPGNPNCLEWLRAKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCTRTTSAVGDVHPGEPVVKDKIKLPTWLGAAITLVVISVIFYFISIYSRPKIKTNDINVRRR

SEQ ID NO: 3 - protein, 377 aa, Tatera poxvirus
MGAAVTLNRIKIAPGIADIRDKYMELGFNYPEYNRVVKFAEESYMYYYETSPGEIKPKFCLIDGMSIDHCSSFIVPEFAKQYVLIHGEPCSSFKFRPGTLIYYQNEVTPEYIKDLKHATDYIASGQRCHFIKKDYLLGDSDSVAKCCSKTNTKHCPKIFNNNYKTEHCDDFMTGFCRNDPGNPNCLEWLRAKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCTRTTSAVGDVHPGEPVVKDKIKLPTWLGAAITLVVISVIFYFISIYSRPKIKTNDINVRRR

SEQ ID NO: 4 - protein, 377 aa, Cowpox virus
MGAAVTLNRIKIAPGIADIRDKYMELGFNYPEYNRAVKFAEESYMYYYETSPGEIKPKFCLIDGMSIDHCSSFIVPEFAKQYVLIRGEPCSSFKFRPGSLIYYQNEVTPEYIKDLKHATDYIASGQRCHFIKKDYLLGDSDSVAKCCSKTNTKHCPKIFNNNYKTEHCDDFMTGFCRNDPGNPNCLEWLRAKRKPAMSTYSDICSKHMDERYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCTRTTSAVGDVHPGEPIVKDKIKLPTWLGAAITLVVISVIFYFISIYSRPKIKTNDINVRRR

SEQ ID NO: 5 - protein, 377 aa, Variola virus
MGAAVTLNRINIASGIADIRDKYMELGFNYPKYNRTVKFAEESYMYYYETSPGEIKPKFCLIDGMSIDHCSSFIVPEFAKQYVLIHGEPCSSFKFRPGTLIYYQNEVTPEYIKDLKHATDYIASGQRCHFIKKDYLLGDSDSVAKCCSKTNTKHCPKIFNNNYKTEHCDDFMTGFCRNDPGNPNCLEWLRVKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCTRTTSTVGDIHPGEPVVKDKIKLPTWLGAAITLVVISVIFYFISIYSRPKIKTNDINVRRR

SEQ ID NO: 6 - protein, 377 aa, Ectromelia virus
MGAAVTLNSIKIETGIADIRDKYMVLGFNYPEYNRAVRFAEESYMYYYETSPGEIKPKFCLIDGMSIDHCSSFIVPEFAKQYVLIRGEPCSSFKFRPGSLIYYQNEVTPEYIKDLKHATDYIASGQRCHFIKKDYLLGDSDSVAKCCSKTNTKHCPKIFNNNYKTEHCDDFMTGFCRNDPGNPNCLEWLRAKRKPAMSTYSDICSKHMDARYCSEFIRIVRPDYFTFGDTALYVFCNDHKGNRNCWCANYPKSNSGDKYLGPRVCWLHECTDESRDRKWLYYNQDVQRTRCKYVGCTVNVNSLALKNSQAELMSNCTRTTSAVGDVHPGEPVVKDKIKLPTWLGAAITLVVISVIFYFISIYSRPKIKTNDINVRRR

SEQ ID NO: 7 - protein, 377 aa, Monkeypox virus
MGAAVTLNRIKIETGIADIRDKYMVLDFNYPEYNRAVRFAEESYMYYYETSPGEIKPKFCLIDGMSIDHCSSFIVPEFAKQYVLIHGEPCSSFKFRPGSLIYYQNEVTPEYIKDLKYATDYIASGQRCHFIKKDYLLGDSDSVAKCCSKTNTKHCPKIFNNNYKTEHCDDFMTGFCRNDPGNPNCLEWLRVKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCTRTTSAVGDVHPGEPVVNDKIKLPTWLGASITLVVISVIFYFISIYSRPKIKTNDINVRRR

SEQ ID NO: 8 - protein, 340 aa, Vaccinia virus
MGGGVSVELPKRDPPPGVPTDEMLLNVDKMHDVIAPAKLLEYVHIGPLAKDKEDKVKKRYPEFRLVNTGPGGLSALLRQSYNGTAPNCCRTFNRTHYWKKDGKISDKYEEGAVLESCWPDVHDTGKCDVDLFDWCQGDTFDRNICHQWIGSAFNRSNRTVEGQQSLINLYNKMQTLCSKDASVPICESFLHHLRAHNTEDSKEMIDYILRQQSADFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFDLKLHLISLLSLLVIWILIVAI

SEQ ID NO: 9 - protein, 340 aa, Tatera poxvirus
MGGGVSVELPKRYPPPGVPTDEMLLNVDKMHDVIAPAKLLEYVHIGPLAKDKEDKVKKRYPEFRLVNTGPGGLSALLRQSYNGTAPNCCRTFNRTHYWKKDGKISDKYEEGAVLESCWPDVHDTGKCDVDLFDWCQGDTFDRNICHQWIGSAFNRSDRTVEGQQSLINLYNKMQTLCSKDASVPICESFLHHLRAHNTEDSKEMIDYILRQQSADFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFDLKLHLISLLSLLVIWILIVAI

SEQ ID NO: 10 - protein, 340 aa, Variola virus
MGGGVSVELPKRDPPPGVPTDEMLLNVDKMHDVIAPAKLLEYVHIGPLAKDKEDKVKKRYPEFRLVNTGPGGLSALLRQSYNGTAPNCCHTFNRTHYWKKDGKISDKYEEGAVLESCWPDVHDTGKCDVNLFDWCQGDTFDRNICHQWIGSAFNRSDRTVEGQQSLINLYNKMQTLCSKDASVPICESFLHHLRAHNTEDSKEMIDYILRQQSANFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFDLKLHLISLLSLLVIWILIVAI

SEQ ID NO: 11 - protein, 340 aa, Monkeypox virus
MGGGVSVELPKRDPPPGVPTDEMLLNVDKMHDVIAPAKLLEYVHIGPLTKDKEDKVKKRYPEFRLVNTGPGGLSALLRQSYNGTAPNCCRTFNRTHYWKKDGKISDKYEEGAVLESCWPDVHDTGKCDVDLFDWCQGDTFDMNICHQWIGSAFNRSDRTVEGRQSLINLYNKMQRLCSKDASVPICELFLHHLRAHNTEDSKEMIDYILRQQSADFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFDLKLHLISLLSLLVIWILIVAI

SEQ ID NO: 12 - protein, 340 aa, Camelpox virus
MGGGVSVELPKRDPPPGVPTDEMLLNVDKMHDVIAPAKLLEYVHIGPLAKDKEDKVKKRYPEFRLVNTGPGGLSALLRQSYNGTTHNCCRTFNRTHYWKKDGKISDKYEEGAVLESCWPDVHDTGKCDVDLFDWCQGDTFDRNICHQWIGSVFNRSDRTVEGQQSLINLYNKMQTLCSKDASVPICESFLHHLRAHNTEDSKEMIDYILRQQSADFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFELKLYLISLLSLLVIWILIVAI

SEQ ID NO: 13 - protein, 340 aa, Cowpox virus
MGGGVSVALPKRDPPPGVPTDEMLLNVDKMHDVIAPAKLLEYVHIGPLAKDKEDKVKKRYPEFRLVSTGPGNLSALLRQSYNGTAPNCCRTFNRTHYWKKDGKISDKYEDGAVLESCWPDVHDTGKCDVDLFDWCQGDTFDRNICHQWIGSAFNRSDRTAEGQQSLINLYNKMQTLCTKDASVPICESFLHHLRAHNTEDSQEMIDYILRQQSADFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFDLKLHLISLLSLLVIWILIVAI

SEQ ID NO: 14 - protein, 340 aa, Ectromelia virus
MGGGVSVALPKRDPPPGVPTDEMLLNVDKMHDVIAPAKLLEYVHIGPLTKDKDDKVKKRYPEFRLVSTGPGNLSALLRQSYNGTAPNCCRTFNRTHYWKKDGKISDKYEDGAELESCWPDAHDTGKCDVDLFDWCQGDTFDRNICHQWIGSAFNRSDRTVDGQQSLINLYNKMQTLCSKDASVPICESFLHHLRAHNTEDSKEMIDYILRQQSTDFKQKYMRCSYPTRDKLEESLKYAEPRECWDPECSNANVNFLLTRNYNNLGLCNIVRCNTSVNNLQMDKTSSLRLSCGLSNSDRFSTVPVNRAKVVQHNIKHSFDLKLHLISLLSLLVIWILIVAI

SEQ ID NO: 15 - protein, 401 aa, synthetic construct
MKLCILLAVVAFVGLSLGRSAAAVTLNRIKIAPGIADIRDKYMELGFNYPEYNRAVKFAEESYTYYYETSPGEIKPKFCLIDGMSIDHCSSFIVPEFAKQYVLIHGEPCSSFKFRPGSLIYYQNEVTPEYIKDLKHATDYIASGQRCHFIKKDYLLGDSDSVAKCCSKTNTKHCPKIFNNNYKTEHCDDFMTGFCRNDPGNPNCLEWLRAKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGCTINVNSLALKNSQAELTSNCARTTSAVGDVHPGEPVVKDKIKGSGLVPRGSGGSGGSHHHHHHHHGGSGTGGLNDIFEAQKIEWHE

SEQ ID NO: 16 - protein, 384 aa, synthetic construct
MKLCILLAVVAFVGLSLGAGGVSVELPKRDPPPGVPTDEMLLNVDKMHDVIAPAKLLEYVHIGPLAKDKEDKVKKRYPEFRLVNTGPGGLSALLRQSYAGTAPNCCRTFQRTHYWKKDGKISDKYEEGAVLESCWPDVHDTGKCDVDLFDWCQGDTFDRNICHQWIGSAFNRADRTVEGQQSLINLYNKMQTLCSKDASVPICESFLHHLRAHNTEDSKEMIDYILRQQSADFKQKYMRCSYPTRDKLEESLKYAEPRECWDPECSNANVNFLLTRNYNNLGLCNIVRCNTNVNNLQMDKTSSLRLSCGLSNSDRFSTVPVNRAKVVQHNIKHSFDLKGSGLVPRGSLEDDDDKAGWSHPQFEKGGGSGGGSGGGSWSHPQFEK

SEQ ID NO: 17 - protein, 358 aa, synthetic construct
MKLCILLAVVAFVGLSLGRSAAAVTLNRIKIAPGIADIRDKYMELGFNYPEYNRAVKFAEESYTYYYETSPGEIKPKFCLIDGMSIDHCSSFIVPEFAKQYVLIHGEPCSSFKFRPGSLIYYQNEVTPEYIKDLKHATDYIASGQRCHFIKKDYLLGDSDSVAKCCSKTNTKHCPKIFNNNYKTEHCDDFMTGFCRNDPGNPNCLEWLRAKRKPAMSTYSDICSKHMDARYCSEFIRIIRPDYFTFGDTALYVFCNDHKGNRNCWCANYPKSNSGDKYLGPRVCWLHECTDESRDRKWLYYNQDVQRTRCKYVGGSGLVPRGSGGSGGSHHHHHHHHGGSGTGGLNDIFEAQKIEWHE

SEQ ID NO: 18 - protein, 334 aa, synthetic construct
MKLCILLAVVAFVGLSLGAGGVSVELPKRDPPPGVPTDEMLLNVDKMHDVIAPAKLLEYVHIGPLAKDKEDKVKKRYPEFRLVNTGPGGLSALLRQSYAGTAPNCCRTFQRTHYWKKDGKISDKYEEGAVLESCWPDVHDTGKCDVDLFDWCQGDTFDRNICHQWIGSAFNRADRTVEGQQSLINLYNKMQTLCSKDASVPICESFLHHLRAHNTEDSKEMIDYILRQQSADFKQKYMRCSYPTRDKLEESLKYAEPRECWDPECSNANVNFLLTRNYNNLGLCNIVRGSGLVPRGSLEDDDDKAGWSHPQFEKGGGSGGGSGGGSWSHPQFEK

SEQ ID NO: 19 - protein, 18 aa, synthetic construct
MKLCILLAVVAFVGLSLG

SEQ ID NO: 20 - protein, 44 aa, synthetic construct
GSGLVPRGSGGSGGSHHHHHHHHGGSGTGGLNDIFEAQKIEWHE

SEQ ID NO: 21 - protein, 46 aa, synthetic construct
GSGLVPRGSLEDDDDKAGWSHPQFEKGGGSGGGSGGGSWSHPQFEK

SEQ ID NO: 22 - protein, 20 aa, synthetic construct
MYRMQLLSCIALSLALVTNS

SEQ ID NO: 23 - DNA, 1209 bp, synthetic construct
  source 1..1209: mol_type = other DNA
atgaagttatgcatattactggccgtcgtggcctttgttggcctctcgctcgggagatctgccgccgccgtgaccctgaaccgcatcaagatcgccccaggcatcgccgatatccgcgacaagtacatggagctgggcttcaactaccccgagtacaatcgcgccgtgaagttcgccgaggagagctacacctactactacgagacctcccccggcgagatcaagccgaagttctgcctgatcgatggcatgtcgatcgaccactgcagctccttcatcgtgccggagttcgccaagcagtacgtgctgatccacggcgagccgtgctcgagcttcaagttccgcccaggcagcctgatctactaccagaacgaggtgaccccagagtacatcaaggatctgaagcacgccaccgactacattgcctcgggacagcgctgccacttcatcaagaaggattacctgctgggcgatagcgactccgtggccaagtgctgctccaagaccaacaccaagcactgccccaagattttcaacaataactacaagaccgagcactgcgatgacttcatgaccggcttctgccgcaatgacccaggcaatcccaactgcctggagtggctgcgcgccaagcgcaagccagccatgtccacctacagcgatatctgctccaagcacatggacgcccgctactgctcggagttcatccgcatcatccgcccggattacttcaccttcggcgacaccgccctgtacgtgttctgcaacgatcacaagggcaatcgcaactgctggtgcgccaactacccgaagtcgaatagcggcgacaagtacctgggaccccgcgtgtgctggctgcacgagtgcaccgatgagtcccgcgaccgcaagtggctgtactacaatcaggatgtgcagcgcacccgctgcaagtacgtgggctgcaccatcaatgtgaacagcctggccctgaagaactcccaggccgagctgacctccaattgcgcccgcaccacctcggccgtgggcgatgtgcacccgggcgagccagtggtgaaggacaagatcaagggcagcggcctggtgccacgcattggaagcggcggctccggcggctcgcaccatcatcaccaccaccaccacggcggctcgggaaccggcggcctgaacgatatcttcgaggcccagaagatcgagtggcacgagtaa

SEQ ID NO: 24 - DNA, 1158 bp, synthetic construct
  source 1..1158: mol_type = other DNA
atgaagttatgcatattactggccgtcgtggcctttgttggcctcagcctaggagccggcggcgtgtcggtggagctgccgaagcgcgatcccccgccaggagtgccgaccgacgagatgctgctgaacgtggataagatgcacgacgtgatcgcccccgccaagctgctggagtacgtgcacatcggcccactggccaaggataaggaggacaaggtgaagaagcgctacccagagttccgcctggtgaacaccggccccggcggcctgtcggccctgctgcgccagtcctacgccggaaccgcccccaattgctgccgcaccttccagcgcacccactactggaagaaggatggcaagatcagcgacaagtacgaggagggagccgtgctggagtcctgctggcccgatgtgcacgacaccggcaagtgcgatgtggacctgttcgattggtgccagggcgataccttcgaccgcaacatctgccaccagtggatcggctcggccttcaatcgcgccgatcgcaccgtggagggacagcagagcctgatcaacctgtacaataagatgcagaccctgtgctccaaggacgcctcggtgccgatctgcgagtccttcctgcaccacctgcgcgcccacaacaccgaggatagcaaggagatgatcgactacatcctgcgccagcagtccgccgatttcaagcagaagtacatgcgctgcagctaccccacccgcgacaagctggaggagtccctgaagtacgccgagccacgcgagtgctgggatccagagtgcagcaatgccaacgtgaatttcctgctgacccgcaactacaataacctgggcctgtgcaatatcgtgcgctgcaacaccaatgtgaataacctgcagatggataagacctcctccctgcgcctgtcctgcggactgagcaactccgatcgcttctcgaccgtgcccgtgaaccgcgccaaggtggtgcagcacaatatcaagcactcgttcgacctgaagggatcgggcctggtgccccgcatcggctccctcgaggacgatgacgataaggccggttggagtcatccacaattcgagaagggcggcggctccggaggtggatcaggaggtggttcctggtcacaccctcaattcgagaagtga

SEQ ID NO: 25 - DNA, 1077 bp, synthetic construct
  source 1..1077: mol_type = other DNA
atgaagttatgcatattactggccgtcgtggcctttgttggcctctcgctcgggagatctgccgccgccgtgaccctgaaccgcatcaagatcgccccaggcatcgccgatatccgcgacaagtacatggagctgggcttcaactaccccgagtacaatcgcgccgtgaagttcgccgaggagagctacacctactactacgagacctcccccggcgagatcaagccgaagttctgcctgatcgatggcatgtcgatcgaccactgcagctccttcatcgtgccggagttcgccaagcagtacgtgctgatccacggcgagccgtgctcgagcttcaagttccgcccaggcagcctgatctactaccagaacgaggtgaccccagagtacatcaaggatctgaagcacgccaccgactacattgcctcgggacagcgctgccacttcatcaagaaggattacctgctgggcgatagcgactccgtggccaagtgctgctccaagaccaacaccaagcactgccccaagattttcaacaataactacaagaccgagcactgcgatgacttcatgaccggcttctgccgcaatgacccaggcaatcccaactgcctggagtggctgcgcgccaagcgcaagccagccatgtccacctacagcgatatctgctccaagcacatggacgcccgctactgctcggagttcatccgcatcatccgcccggattacttcaccttcggcgacaccgccctgtacgtgttctgcaacgatcacaagggcaatcgcaactgctggtgcgccaactacccgaagtcgaatagcggcgacaagtacctgggaccccgcgtgtgctggctgcacgagtgcaccgatgagtcccgcgaccgcaagtggctgtactacaatcaggatgtgcagcgcacccgctgcaagtacgtgggcggcagcggcctggtgccacgcggaagcggcggctccggcggctcgcaccatcatcaccaccaccaccacggcggctcgggaaccggcggcctgaacgatatcttcgaggcccagaagatcgagtggcacgagtaa

SEQ ID NO: 26 - DNA, 1005 bp, synthetic construct
  source 1..1005: mol_type = other DNA
atgaagttatgcatattactggccgtcgtggcctttgttggcctcagcctaggagccggcggcgtgtcggtggagctgccgaagcgcgatcccccgccaggagtgccgaccgacgagatgctgctgaacgtggataagatgcacgacgtgatcgcccccgccaagctgctggagtacgtgcacatcggcccactggccaaggataaggaggacaaggtgaagaagcgctacccagagttccgcctggtgaacaccggccccggcggcctgtcggccctgctgcgccagtcctacgccggaaccgcccccaattgctgccgcaccttccagcgcacccactactggaagaaggatggcaagatcagcgacaagtacgaggagggagccgtgctggagtcctgctggcccgatgtgcacgacaccggcaagtgcgatgtggacctgttcgattggtgccagggcgataccttcgaccgcaacatctgccaccagtggatcggctcggccttcaatcgcgccgatcgcaccgtggagggacagcagagcctgatcaacctgtacaataagatgcagaccctgtgctccaaggacgcctcggtgccgatctgcgagtccttcctgcaccacctgcgcgcccacaacaccgaggatagcaaggagatgatcgactacatcctgcgccagcagtccgccgatttcaagcagaagtacatgcgctgcagctaccccacccgcgacaagctggaggagtccctgaagtacgccgagccacgcgagtgctgggatccagagtgcagcaatgccaacgtgaatttcctgctgacccgcaactacaataacctgggcctgtgcaatatcgtgcgcggatcgggcctggtgccccgcggctccctcgaggacgatgacgataaggccggttggagtcatccacaattcgagaagggcggcggctccggaggtggatcaggaggtggttcctggtcacaccctcaattcgagaagtga